SEQUENCE LISTING (12 sequences)

SEQ ID NO: 1 - protein, 801 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 2 - protein, 444 aa, synthetic construct
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIQMTQSPSSLSASVGDRVTITCKASQGVSNDVAWYQQKPGKAPKLLIYSASYRYTGVPSRFSGSGSGTDFTFTISSLQPEDIATYYCQQHSTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 114 aa, synthetic construct [sequence omitted]

SEQ ID NO: 5 - protein, 107 aa, synthetic construct [sequence omitted]

SEQ ID NO: 6 - protein, 5 aa, synthetic construct
TYNVH

SEQ ID NO: 7 - protein, 17 aa, synthetic construct
SIYPDNGDTSYNQNFKG

SEQ ID NO: 8 - protein, 5 aa, synthetic construct
GDFAY

SEQ ID NO: 9 - protein, 11 aa, synthetic construct
KASQGVSNDVA

SEQ ID NO: 10 - protein, 7 aa, synthetic construct
SASYRYT

SEQ ID NO: 11 - protein, 9 aa, synthetic construct
QQHSTTPYT

SEQ ID NO: 12 - protein, 800 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

(19) United States
(12) Patent Application Publication
(10) Pub. No.: US 2025/0243284 A1
(43) Pub. Date: Jul. 31, 2025

(54) TREATMENT OF SOLID TUMORS

(71) Applicant: AMGEN INC., Thousand Oaks, CA (US)

(72) Inventors: Xiaojun HUANG, Thousand Oaks, CA (US); Alissa KEEGAN, Thousand Oaks, CA (US); Leticia Charis' ARRINGTON, Thousand Oaks, CA (US); Sumera RAOOF, Thousand Oaks, CA (US)

(73) Assignee: AMGEN INC., Thousand Oaks, CA (US)

(21) Appl. No.: 18/854,646

(22) PCT Filed: Apr. 6, 2023
(86) PCT No.: PCT/US2023/065418

§ 371 Date: Oct. 7, 2024

Related U.S. Application Data
(60) Provisional application No. 63/328,789, filed on Apr. 8, 2022.
(60) Provisional application No. 63/377,266, filed on Sep. 27, 2022.
(60) Provisional application No. 63/492,047, filed on Mar. 24, 2023.

(51) Int. Cl. C07K 16/28; A61K 9/00; A61K 39/00; A61P 35/00; G01N 33/574

(52) U.S. Cl. CPC C07K 16/2863; A61K 9/0019; A61P 35/00; G01N 33/57492; A61K 2039/505; A61K 2039/54; A61K 2039/545; C07K 2317/41; C07K 2317/565; G01N 2333/70532; G01N 2333/71; G01N 2474/20

(57) ABSTRACT

Methods of treating solid tumors, such as squamous cancer (such as head and neck squamous cell carcinoma), ER− PR− HER2/neu− (“triple-negative”) breast cancer, intrahepatic cholangiocarcinoma, lung adenocarcinoma, and gynecological malignancy, in subjects are described. The methods may comprise administering an anti-FGFR2b antibody to the subject.

### FIELD

Embodiments herein relate to uses of antibodies against fibroblast growth factor 2 (FGFR2), including antibodies against the FGFR2 isoform FGFR2-IIIb (also referred to as FGFR2b), in the treatment of solid tumors, such as, for example, squamous cancer (such as head and neck squamous cell carcinoma), “triple-negative” breast cancer, intrahepatic cholangiocarcinoma, lung adenocarcinoma, and gynecological malignancy.

### INCORPORATION-BY-REFERENCE OF MATERIAL SUBMITTED ELECTRONICALLY

Incorporated by reference in its entirety herein is a nucleotide/amino acid sequence listing submitted concurrently herewith and identified as follows: One 13,839 byte XML document named “10131-WO01-SEC.xml,” created on Mar. 27, 2023.

### CROSS-REFERENCE TO RELATED APPLICATIONS

This application claims the benefit of U.S. Provisional Application No. 63/328,789, filed Apr. 8, 2022; U.S. Provisional Application No. 63/377,266, filed Sep. 27, 2022; and U.S. Provisional Application No. 63/492,047, filed Mar. 24, 2023, the disclosures of which are incorporated by reference herein.

### BACKGROUND

Fibroblast growth factor receptor 2b (FGFR2b) is overexpressed in a subset of gastric/gastroesophageal junction (GC/GEJ) cancers and is a target for new therapies that may improve treatment outcomes. The fibroblast growth factor (FGF) family members bind to four known tyrosine kinase receptors, fibroblast growth factor receptors 1-4 (FGFR1 4) and their isoforms, with the various FGFs binding the different FGFRs to varying extents (Zhang et al., J. Biol. Chem. 281:15694, 2006). A protein sequence of human FGFR2 is provided in, e.g., GenBank Locus AF487553. Each FGFR consists of an extracellular domain (ECD) comprising three immunoglobulin (Ig)-like domains (D1, D2 and D3), a single transmembrane helix, and an intracellular catalytic kinase domain (Mohammadi et al., Cytokine Growth Factor Revs, 16:107, 2005). FGFs bind to the receptors primarily through regions in D2 and D3 of the receptors. There is a contiguous stretch of acidic amino acids in the linker between D1 and D2 called the “acid box” (AB). The region containing D1 and AB is believed to be involved in autoinhibition of the receptor, which is relieved by binding to ligand.

The FGFRs are characterized by multiple alternative splicing of their mRNAs, leading to a variety of isoforms (Ornitz et al., J. Biol. Chem. 271:15292, 1996; see also Swiss-Prot P21802 and isoforms P21802-1 to -20 for sequences of FGFR2 and its isoforms). Notably, there are forms containing all three Ig domains (α isoform) or only the two Ig domains D2 and D3 domains without D1 (β isoform). In FGFR1, FGFR2, and FGFR3, all forms contain the first half of D3 denoted IIIa, but two alternative exons can be utilized for the second half of D3, leading to IIIb and IIIc forms. For FGFR2, these are respectively denoted FGFR2-IIIb and FGFR2-IIIc (or just FGFR2b and FGFR2c, respectively); the corresponding beta forms are denoted FGFR2(beta)IIIb and FGFR2(beta)IIIc. The FGFR2-IIIb form of FGFR2 (also denoted K-sam-II) is a high affinity receptor for both FGF1 and KGF family members (FGF7, FGF10, and FGF22) whereas FGFR2-IIIc (also denoted K-sam-I) binds both FGF1 and FGF2 well but does not bind the KGF family members (Miki et al., Proc. Natl. Acad. Sci. USA 89:246, 1992). Indeed, FGFR2-IIIb is the only receptor for KGF family members (Ornitz et al., supra) and is therefore also designated KGFR.

The FGFRs and their isoforms are differentially expressed in various tissues. FGFR2-IIIb (and the IIIb forms of FGFR1 and FGFR3) is expressed in epithelial tissues, while FGFR2-IIIc is expressed in mesenchymal tissues (Duan et al., J. Biol. Chem. 267:16076, 1992; Ornitz et al., 1996, supra). Certain of the FGF ligands of these receptors have an opposite pattern of expression. Thus, KGF subfamily members, including FGF7 (KGF), FGF10, and FGF22, bind only to FGFR2-IIIb (Zhang et al., supra) and are expressed in mesenchymal tissues, and so may be paracrine effectors of epithelial cells (Ornitz et al., supra). In contrast, the FGF4 subfamily members FGF4-6 bind to FGFR2-IIIc and are expressed in both epithelial and mesenchymal lineages, and so may have either autocrine or paracrine functions. Because of the expression patterns of the isoforms of FGFR2 and their ligands, FGFR2 plays a role in epithelial-mesenchymal interactions (Finch et al., Dev. Dyn. 203:223, 1995), and knock-out of FGFR2-IIIb in mice leads to severe embryonic defects and lethality (De Moerlooze et al., Development 127:483, 2000).

Besides gastric cancer, other epithelial malignancies have been reported to overexpress FGFR2 at the DNA or protein level, including head and neck squamous cell carcinoma (Brands et al, 2017), esophageal carcinoma (Kato et al, 2013), colorectal cancer (Li et al, 2019), intrahepatic cholangiocarcinoma (Churi et al, 2014), pancreatic cancer (Kang et al, 2019), breast cancer (Reinjtes et al, 2013), ovarian cancer (Tyulyandina et al, 2018), cervical cancer (Sun et al, 2019) endometrial cancer (Gatius et al, 2011; Dutt et al, 2008), and others (Wu et al, 2013). Treatment of such malignancies typically involves the use of multiple modalities, such as surgery, systemic anti-cancer therapy (SACT) and radiotherapy, alone or in combination or sequentially. There remains a need for effective monotherapies for certain types of solid cancers.

### SUMMARY

The disclosure provides a method of treating a solid tumor in a subject, comprising administering to the subject an anti-FGFR2b antibody monotherapy comprising either: (a) an every two weeks (Q2W) regimen of a first administration of the anti-FGFR2b antibody at a dose of greater than 20 mg/kg to no more than 30 mg/kg, followed two weeks after the first administration and Q2W thereafter by subsequent administrations of the anti-FGFR2b antibody each at a dose of 12-20 mg/kg, wherein the subsequent administrations are at a lower dose than the first administration; or (b) an every two weeks (Q2W) regimen of the anti-FGFR2b antibody at a dose of greater than 10 mg/kg to no more than 20 mg/kg, and one week after the first administration of the anti-FGFR2b antibody, administering a single subsequent administration of the anti-FGFR2b antibody at a dose of 5-10 mg/kg.

In some embodiments of the above method, or any method of treating a solid tumor herein, the solid tumor is selected from the group consisting of squamous cancer (such as head and neck squamous cell carcinoma), ER− PR− HER2/neu− (“triple-negative”) breast cancer, pancreatic ductal adenocarcinoma, intrahepatic cholangiocarcinoma, colorectal adenocarcinoma, and gynecological malignancy.

In some embodiments of the above method, or any method of treating a solid tumor herein, the solid tumor is selected from the group consisting of squamous cancer (such as head and neck squamous cell carcinoma), “triple-negative” breast cancer, intrahepatic cholangiocarcinoma, lung adenocarcinoma, and gynecological malignancy.

In some embodiments of the above method, or any method of treating a solid tumor herein, the anti-FGFR2b antibody monotherapy is administered as a second line or beyond therapy for the solid tumor, such as third line or beyond.

In some embodiments of the above method, or any method of treating a solid tumor herein, the squamous cancer is head and neck cancer or squamous esophageal cancer. In some embodiments of the above method, or any method of treating a solid tumor herein the squamous cancer is head and neck squamous cell carcinoma.

In some embodiments of the above method, or any method of treating a solid tumor herein, the gynecological malignancy is selected from the group consisting of ovarian epithelial cancer (including fallopian tube cancer and primary peritoneal cancer), endometrial cancer, and cervical cancer.

In some embodiments of the above method, or any method of treating a solid tumor herein, the squamous cancer is post platinum-based chemotherapy and/or post-PD-1 inhibitor.

In some embodiments of the above method, or any method of treating a solid tumor herein, the triple negative breast cancer is post chemotherapy, post-PARPi (if BRCA-mutated), post-PD-1 inhibitor therapy, and/or post-anti-trop-2 therapy.

In some embodiments of the above method, or any method of treating a solid tumor herein, the pancreatic ductal adenocarcinoma is post-platinum based chemotherapy, the intrahepatic cholangiocarcinoma is post-platinum based chemotherapy and post-targeted therapy, if eligible for targeted therapy, and/or the colorectal adenocarcinoma is post-bevacizumab therapy, post-oxaliplatin-based chemotherapy, post-irinotecan-based chemotherapy, and/or post-additional prior therapy based on RAS, BRAF, and dMVMR/MSI-H status.

In some embodiments of the above method, or any method of treating a solid tumor herein, the gynecological malignancy is post platinum-based chemotherapy, and/or is platinum chemotherapy resistant.

In some embodiments of the above method, or any method of treating a solid tumor herein, the cells of the solid tumor overexpress FGFR2b mRNA or protein, or comprise an FGFR2 gene amplification.

In some embodiments of the above method, or any method of treating a solid tumor herein, the solid tumor overexpresses FGFR2b as determined by immunohistochemistry (IHC).

In some embodiments of the above method, or any method of treating a solid tumor herein, cells of the solid tumor are positive for FGFR2b as determined by IHC, optionally wherein at least 5%, 10%, or 20% of the cells are positive for FGFR2b.

In some embodiments of the above method, or any method of treating a solid tumor herein, cells of the solid tumor exhibit 2+ and/or 3+ FGFR2b staining as determined by IHC, optionally wherein at least 5%, 10%, or 20% of the cells exhibit said FGFR2b staining.

In some embodiments of the above method, or any method of treating a solid tumor herein, cells of the solid tumor are PD-L1 positive, as determined by IHC staining.

In some embodiments of the above method, (a) the first administration of the anti-FGFR2b antibody is at a dose of greater than 20 mg/kg to no more than 25 mg/kg, followed two weeks after the first administration and Q2W thereafter by subsequent administrations of the anti-FGFR2b antibody each at a dose of 12-17 mg/kg.

In some embodiments of the above method, or any method of treating a solid tumor herein, (a) the first administration of the anti-FGFR2b antibody is at a dose of 22-25 mg/kg, followed two weeks after the first administration and Q2W thereafter by subsequent administrations of the anti-FGFR2b antibody each at a dose of 12-17 mg/kg.

In some embodiments of the above method, or any method of treating a solid tumor herein, (a) the first administration of the anti-FGFR2b antibody is at a dose of about 22 mg/kg, followed two weeks after the first administration and Q2W thereafter by subsequent administrations of the anti-FGFR2b antibody each at a dose of about 15 mg/kg.

In some embodiments of the above method, or any method of treating a solid tumor herein, (b) the Q2W regimen of the anti-FGFR2b antibody is at a dose of 12-17 mg/kg, and the subsequent single administration of the anti-FGFR2b antibody one week after the first administration of the anti-FGFR2b antibody is at a dose of 7-8 mg/kg.

In some embodiments of the above method, or any method of treating a solid tumor herein, (b) the Q2W regimen of the anti-FGFR2b antibody is at a dose of about 15 mg/kg, and the subsequent single administration of the anti-FGFR2b antibody one week after the first administration of the anti-FGFR2b antibody is at a dose of about 7.5 mg/kg.

In some embodiments of the above method, or any method of treating a solid tumor herein, the anti-FGFR2b antibody is administered intravenously.

In some embodiments of the above method, or any method of treating a solid tumor herein, the anti-FGFR2b antibody comprises a heavy chain variable region comprising a heavy chain complementarity determining region (HCDR) 1 of SEQ ID NO: 6, a HCDR2 of SEQ ID NO: 7, and a HCDR3 of SEQ ID NO: 8; and a light chain variable region comprising a light chain complementarity determining region (LCDR) 1 of SEQ ID NO: 9, a LCDR2 of SEQ ID NO: 10, and a LCDR3 of SEQ ID NO: 11.

In some embodiments of the above method, or any method of treating a solid tumor herein, the anti-FGFR2b antibody is afucosylated.

In some embodiments of the above method, or any method of treating a solid tumor herein, the heavy chain variable region of the anti-FGFR2b antibody comprises an amino acid sequence at least 95% identical to SEQ ID NO: 4, and the light chain variable region of the anti-FGFR2b antibody comprises an amino acid sequence at least 95% identical to SEQ ID NO: 5.

In some embodiments of the above method, or any method of treating a solid tumor herein, the heavy chain variable region of the anti-FGFR2b antibody comprises the amino acid sequence of SEQ ID NO: 4, and the light chain variable region of the anti-FGFR2b antibody comprises the amino acid sequence of SEQ ID NO: 5.

In some embodiments of the above method, or any method of treating a solid tumor herein, the anti-FGFR2b antibody comprises the heavy chain of SEQ ID NO: 1, the light chain of SEQ ID NO: 2, and the anti-FGFR2b antibody lacks fucose at Asn297 (EU numbering).

In some embodiments of the above method, or any method of treating a solid tumor herein, the anti-FGFR2b antibody is bemarituzumab.

In some embodiments of the above method, or any method of treating a solid tumor herein, the bemarituzumab is administered intravenously, and (a) the first administration of the bemarituzumab is at a dose of greater than 20 mg/kg to no more than 25 mg/kg, followed two weeks after the first administration and Q2W thereafter by subsequent administrations each at a dose of 12-17 mg/kg.

In some embodiments of the above method, or any method of treating a solid tumor herein, the bemarituzumab is administered intravenously, and (a) the first administration of the bemarituzumab is at a dose of 22-25 mg/kg, followed two weeks after the first administration and Q2W thereafter by subsequent administrations each at a dose of 12-17 mg/kg.

In some embodiments of the above method, or any method of treating a solid tumor herein, the bemarituzumab is administered intravenously, and (a) the first administration is at a dose of about 22 mg/kg, followed two weeks after the first administration and Q2W thereafter by subsequent administrations of the bemarituzumab each at a dose of about 15 mg/kg.

In some embodiments of the above method, or any method of treating a solid tumor herein, the bemarituzumab is administered intravenously, and (b) the Q2W regimen of the bemarituzumab is at a dose of 12-17 mg/kg, and the subsequent single administration of the bemarituzumab one week after the first administration is at a dose of 7-8 mg/kg.

In some embodiments of the above method, or any method of treating a solid tumor herein, the bemarituzumab is administered intravenously, and (b) the Q2W regimen of the bemarituzumab is at a dose of about 15 mg/kg, and the subsequent single administration of the bemarituzumab one week after the first administration is at a dose of about 7.5 mg/kg.

### BRIEF DESCRIPTION OF THE DRAWINGS

FIGS. 1A-1B are diagrams of methods of treating various solid tumor types.

FIGS. 2A-2B are diagrams of amino acid sequences. FIG. 2A depicts amino acid sequences of anti-FGFR2b antibodies of some embodiments. FIG. 2B depicts amino acid sequences of FGFR2s of some embodiments.

FIGS. 3A-3I are graphs showing ADCC response of solid tumor cells treated with bemarituzumab in accordance with some embodiments.

FIG. 4 is a schematic of the Schedule of Activities for the study described in Example 2.

### DETAILED DESCRIPTION

Described herein are methods of treating a solid tumor in a subject. The cells of the solid tumor may overexpress FGFR2 isoform FGFR2-IIIb (also known as FGFR2b). The methods can comprise administering an anti-FGFR2b antibody such as bemarituzumab to the subject. The methods can comprise administration of an anti-FGFR2b antibody as a monotherapy (e.g., as a single therapeutic). It is contemplated herein that the anti-FGFR2b antibody monotherapy may be administered via two possible dosing regimens. For example, one anti-FGFR2b antibody monotherapy may comprise an every two weeks (Q2W) regimen of a first administration of the anti-FGFR2b antibody at a dose of greater than about 20 mg/kg and no more than about 30 mg/kg, such as 20-25 mg/kg, 21-30 mg/kg or 22-25 mg/kg or 25-30 mg/kg, followed two weeks after the first administration and Q2W thereafter by subsequent administrations of the anti-FGFR2b antibody each at a dose of about 12-20 mg/kg, such as about 12-17 mg/kg (e.g., 12 mg/kg, 13 mg/kg, 14 mg/kg, 15 mg/kg, 16 mg/kg, or 17 mg/kg), about 15-17 mg/kg, or about 18 mg/kg, about 19 mg/kg, or about 20 mg/kg. For example, the anti-FGFR2b antibody may be administered Q2W to the subject in a first administration at a dose of greater than 20 mg/kg to no more than 25 mg/kg, such as 22-25 mg/kg, followed two weeks after the first administration and Q2W thereafter by subsequent administrations each at a dose of 12-17 mg/kg, such as 15-17 mg/kg. For example, the anti-FGFR2b antibody may be administered Q2W to the subject in a first administration at a dose of 22-25 mg/kg, such as 22 mg/kg, 23 mg/kg, 24 mg/kg, or 25 mg/kg, followed two weeks after the first administration and Q2W thereafter by subsequent administrations each at a dose of 15-17 mg/kg, such as 15 mg/kg, 16 mg/kg, or 17 mg/kg. For example, the anti-FGFR2b antibody may be administered Q2W to the subject in a first administration at a dose of about 22 mg/kg, followed two weeks after the first administration and Q2W thereafter by subsequent administrations each at a dose of about 15 mg/kg.

A further anti-FGFR2b antibody monotherapy may comprise an every two weeks (Q2W) regimen of the anti-FGFR2b antibody at a dose of greater than about 10 mg/kg and no more than about 20 mg/kg of the anti-FGFR2b antibody, such as 10 mg/kg, 11 mg/kg, 12 mg/kg, 13 mg/kg, 14 mg/kg, 15 mg/kg, 16 mg/kg, 17 mg/kg 18 mg/kg, 19 mg/kg, or 20 mg/kg, and one week after the first administration of the anti-FGFR2b antibody, administering a single subsequent administration of the anti-FGFR2b antibody at a dose of about 5-10 mg/kg, such as 5 mg/kg, 6 mg/kg, 7 mg/kg, 8 mg/kg, 9 mg/kg, or 10 mg/kg. For example, the Q2W regimen of the anti-FGFR2b antibody is at a dose of 12-17 mg/kg (e.g., 15-17 mg/kg), and the subsequent single administration of the anti-FGFR2b antibody one week after the first administration of the anti-FGFR2b antibody is at a dose of 7-8 mg/kg. For example, the Q2W regimen of the anti-FGFR2b antibody is at a dose of about 15 mg/kg, and the subsequent single administration of the anti-FGFR2b antibody one week after the first administration of the anti-FGFR2b antibody is at a dose of about 7.5 mg/kg.

Alterations in the FGF/FGFR2 signaling pathway have been reported in the literature for multiple tumor types. Data from The Cancer Genome Atlas (TCGA), report that detection of FGFR2 overexpression by mRNA is common amongst epithelial cancers. Among high expressing cancers (fragments per kilobase of transcript per million [FPKM]>10) are cholangiocarcinoma (93.3%), ovarian cancer (78.8%), pancreatic (33.9%), triple negative breast cancer (41.8%), head and neck carcinoma (57.5%), esophageal squamous cell carcinoma (45.7%), endometrial carcinoma (46.7%), cervical cancer (29.4%), and colorectal cancer (12.9%). Immunohistochemistry (IHC) suggests overexpression (as subjects with FGFR2b 2+/3+ membrane staining in tumor cells) in ovarian carcinoma (19%, 95% CI 14%-24%), endometrial carcinoma (23%, 95% CI 17%-29%), squamous cell carcinoma of the head and neck (22%, 95% CI 15%-29%), cervical carcinoma (10%, 95% CI 4%-16%), triple negative breast cancer (8%, 95% CI 4%-13%), adenocarcinoma of the lung (6%, 95% CI 1%-11%), and intrahepatic cholangiocarcinoma (iCCA, 1%, 95% CI 0%-3%) (Table 10). Evaluation of bemarituzumab in subjects with FGFR2b overexpressing tumors may improve the outcome for these subjects by providing targeted inhibition of tumor growth signaling.

### Anti-FGFR2b Antibodies

As used herein, “antigen binding protein” has its customary and ordinary meaning as understood by one of ordinary skill in the art in view of this disclosure. It refers to a protein that specifically binds a specified antigen. The term encompasses intact antibodies as well as derivatives, variants, fragments, and mutants thereof. An antigen binding protein also includes bivalent and polyvalent/multivalent constructs as well as bispecific and polyspecific/multispecific constructs, as well as domain antibodies, scFvs, and both membrane-bound and soluble receptors. In some embodiments, an antigen binding protein comprises, consists essentially of, or consists of an antibody. In any of the methods described herein, an anti-FGFR2b antigen binding protein may be administered to the subject. By way of example, the antigen binding protein may comprise or consist of an antibody, for example bemarituzumab.

An antibody is an example of an antigen binding protein. As used herein, “antibody” has its customary and ordinary meaning as understood by one of ordinary skill in the art in view of this disclosure. It refers to an immunoglobulin of any isotype with specific binding to the target antigen, and includes, for instance, chimeric, humanized, fully human, and monoclonal antibodies. An “antibody” as such is a subgenus of an antigen binding protein. For example, human or humanized antibodies can be of any isotype, including IgG (including IgG1, IgG2, IgG3 and IgG4 subtypes), IgA (including IgA1 and IgA2 subtypes), IgM and IgE. A human IgG antibody generally will comprise two full-length heavy chains and two full-length light chains. Antibodies may be derived solely from a single source, or may be “chimeric,” that is, different portions of the antibody may be derived from two or more different antibodies from the same or different species. It will be understood that once an antibody is obtained from a source, it may undergo further engineering, for example to enhance stability and folding. Accordingly, it will be understood that a “human” antibody may be obtained from a source, and may undergo further engineering, for example in the Fc region. The engineered antibody may still be referred to as a type of human antibody. Similarly, variants of a human antibody, for example those that have undergone affinity maturation, will also be understood to be “human antibodies” unless stated otherwise. In some embodiments, the antigen binding protein comprises, consists essentially of, or consists of a human, humanized, or chimeric monoclonal antibody.

A “heavy chain” of an antigen binding protein (such as an antibody) includes a variable region (“VHI”), and three constant regions: CH1, CH2, and CH3. A “light chain” of an antigen binding protein (such as an antibody) includes a variable region (“VL”), and a constant region (“CL”). Human light chains include kappa chains and lambda chains.

“Antigen binding region” means a protein, or a portion of a protein, that specifically binds a specified antigen. For example, that portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”) of an antibody. A “CDR” is an amino acid sequence that contributes to antigen binding specificity and affinity. Antigen binding regions of antibody heavy and light chains generally exhibit the same overall structure, comprising relatively conserved framework regions (FR) joined by three CDRs. The CDRs from the two chains of each heavy chain/light chain pair typically are aligned by the framework regions to form a structure that binds specifically with a specific epitope on the target protein. From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, National Institutes of Health, Bethesda, Md.), or Chothia & Lesk, 1987, J. Mol. Biol. 196: 901-917; Chothia et al., 1989, Nature 342: 878-883. In some embodiments, the CDRs of an antigen binding protein are defined according to the definition of Kabat or Chothia.

Antigen binding proteins against FGFR2b, such as antibodies, may be used in methods described herein. The antibodies may specifically bind to FGFR2b. In the method of some embodiments, the anti-FGFR2b antigen binding protein binds with a higher affinity to FGFR2b than to FGFR2-IIIc. For example, the anti-FGFR2b antibodies may not detectably bind to FGFR-IIIc. In the method of some embodiments, anti-FGFR2b antigen binding protein (e.g., antibody) binds to FGFR2b and blocks or inhibits signaling through the FGFR2b receptor. For example, the binding of the anti-FGFR2b antigen binding protein (e.g., antibody) to FGFR2b may inhibit phosphorylation of FGFR2 or a MAP kinase downstream of FGFR2. In the method of some embodiments, the anti-FGFR2b antigen binding protein (e.g., antibody), upon binding to FGFR2b, inhibits binding between FGFR2b and an FGF ligand thereof, such as FGF1 and/or FGF2.

Binding of antigen binding protein (e.g., antibody) to FGFR2b and inhibition of binding between FGFR2b and FGFs can be assessed, for example, by ELISA assays, as described in U.S. Pat. No. 8,101,723, or, for example, by a chip-based assay as described in Example 2 of WO 2015/017600. In some embodiments, the antibody induces an ADCC activity, and in some embodiments possesses enhanced ADCC activity, for example, as described in WO 2015/017600. ADCC activity, for example, may be determined as described in Example 3 of WO 2015/07600. In some embodiments, the antibody may inhibit growth of a human tumor in a mouse model, for example, as shown in Example 1 of WO 2017/091577. In some embodiments, the anti-FGFR2-IIIb antibody is capable of increasing the number of one or more of PD-L1 positive cells, NK cells, CD3+ T cells, CD4+ T cells, CD8+ T cells, and macrophages in tumor tissue in a mouse tumor model compared to a control, for example, as described in Example 2 of International Application No. WO 2017/091577.

Any of the anti-FGFR2b antibodies described herein may be afucosylated. For example, the antibody may be an IgG1 or IgG3 antibody that lacks fucose at Asn297. As used herein, an “afucosylated” antibody or an antibody “lacking fucose” refers to an IgG1 or IgG3 isotype antibody that lacks fucose in its constant region glycosylation. Glycosylation of human IgG1 or IgG3 occurs at Asn297 (N297; EU number of Fc region residue) as core fucosylated biantennary complex oligosaccharide glycosylation terminated with up to 2 Gal residues. In some embodiments, an afucosylated antibody lacks fucose at Asn297. These structures are designated as G0, G1 (α1,6 or α1,3) or G2 glycan residues, depending on the amount of terminal Gal residues. See, e.g., Raju, T. S., BioProcess Int. 1: 44-53 (2003). CHO type glycosylation of antibody Fc is described, e.g., in Routier, F. H., Glycoconjugate J. 14: 201-207 (1997). It will be appreciated that compositions comprising monoclonal antibodies are often heterogenous. As a practical matter, methods comprising administration of an afucosylated anti-FGFR2 antibody described herein may further comprise administering some antibody molecules that are not afucosylated. Within a population of antibodies, the antibodies are considered to be afucosylated if <5% of the antibodies of the population comprise fucose at Asn297. For example, in some embodiments, greater than 95% of the molecules of anti-FGFR2b antibody administered to the subject are afucosylated. For example, in some embodiments, at least 96%, 97%, or 99% of the molecules of anti-FGFR2b antibody administered to the subject may be afucosylated. Additional antibodies that may be used in embodiments herein include those described in US Patent Publication No. 2015/0050273, which describes certain afucosylated anti-FGFR2b antibodies, and which is incorporated herein by reference in its entirety.

In some embodiments, an afucosylated anti-FGFR2b antibody mediates antibody-dependent cell-mediated cytotoxicity (ADCC) in the presence of human effector cells more effectively than an antibody with the same amino acid sequence that comprises fucose. Generally, ADCC activity may be determined using the in vitro ADCC assay disclosed in U.S. Patent Publication No. 2015/0050273, but other assays or methods for determining ADCC activity, e.g. in an animal model etc., are contemplated.

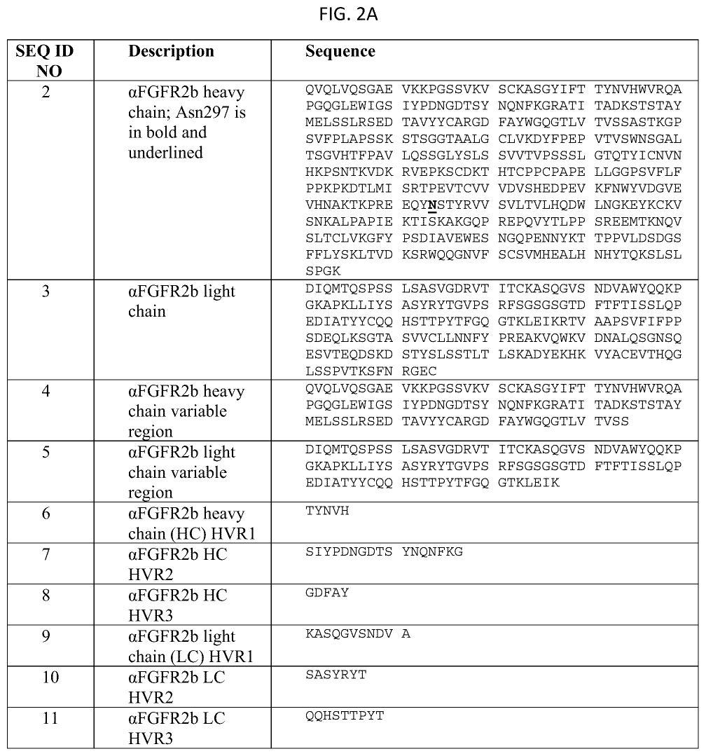

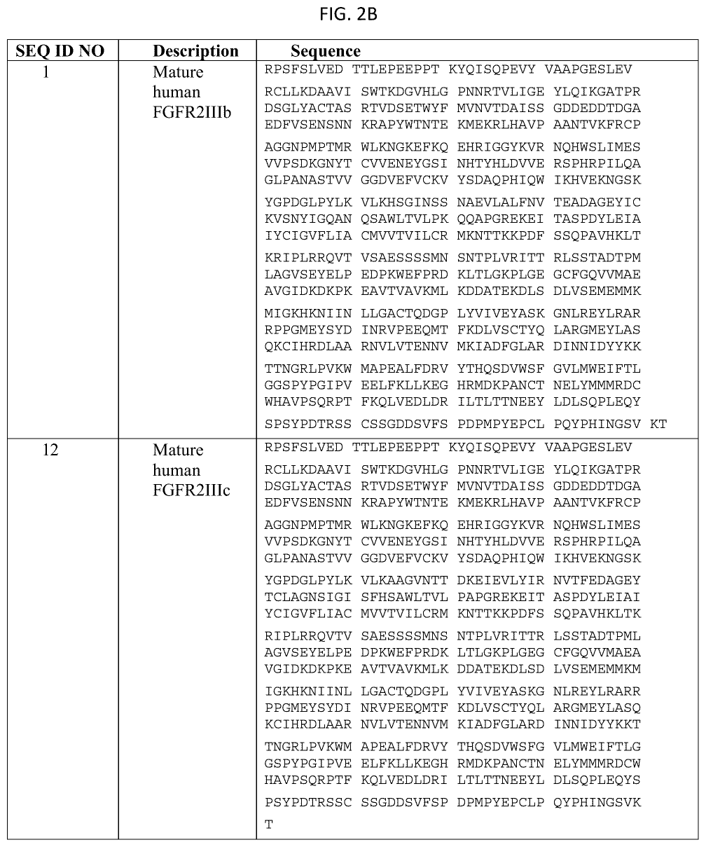

Example sequences of anti-FGFR2b antibodies of some embodiments are shown in FIG. 2A. In the method of some embodiments, the anti-FGFR2b antibody comprises at least one, two, three, four, five, or six complementarity determining regions (CDRs) selected from (a) a HCDR1 of SEQ ID NO: 6; (b) a HCDR2 of SEQ ID NO: 7; (c) a HCDR3 of SEQ ID NO: 8; (d) a LCDR1 of SEQ ID NO: 9; (e) a LCDR2 of SEQ ID NO: 10; and (f) a LCDR3 of SEQ ID NO: 11. The anti-FGFR2b may comprise a heavy chain comprising a heavy chain variable region comprising a HCDR1 of SEQ ID NO: 6, a HCDR2 of SEQ ID NO: 7, and a HCDR3 of SEQ ID NO: 8, and may further comprise a light chain comprising a light chain variable region comprising a LCDR1 of SEQ ID NO: 9, a LCDR2 of SEQ ID NO: 8, and a LCDR3 of SEQ ID NO: 9. In the method of some embodiments, the heavy chain variable region is at least 90% identical to SEQ ID NO: 4 and the light chain variable region is at least 90% identical to SEQ ID NO: 5. In the method of some embodiments, the heavy chain variable region is at least 95% identical to SEQ ID NO: 4 and the light chain variable region is at least 95% identical to SEQ ID NO: 5. In some embodiments, the heavy chain variable region comprises SEQ ID NO: 4 and the light chain variable region comprises SEQ ID NO: 5. In the method of some embodiments, the heavy chain comprises SEQ ID NO: 2 and the light chain comprises SEQ ID NO: 3. Any of the anti-FGFR2 antibodies described herein may be afucosylated. For example, the antibody may be an IgG1 or IgG3 antibody that lacks fucose at Asn297. In the method of some embodiments, the anti-FGFR2b antibody is bemarituzumab.

In the method of some embodiments, the anti-FGFR2b antibody comprises a heavy chain variable region comprising SEQ ID NO: 4 and a light chain variable region comprising SEQ ID NO: 5. It is further contemplated that in some embodiments, the anti-FGFR2b antibody comprises one or more substitutions, insertions, or deletions compared to SEQ ID NO: 4 and/or SEQ ID NO: 5, and continues to bind to FGFR2b. For example, the anti-FGFR2b antibody comprises one or more substitutions, insertions, or deletions compared to SEQ ID NO: 4 and/or SEQ ID NO: 5 and may bind to FGFR2b with an affinity, as measured by surface plasmon resonance, that is no less than an order of magnitude lower than the affinity of a reference anti-FGFR2b antibody comprising a heavy chain variable region comprising SEQ ID NO: 4 and a light chain variable region comprising SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 90% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 91% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 95% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 97% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region of SEQ ID NO: 5. In the method of some embodiments, the anti-FGFR2b antibody comprises a heavy chain variable region at least 90% identical to SEQ ID NO: 4 and a light chain variable region at least 90% identical to SEQ ID NO: 5. In the method of some embodiments, the heavy chain variable region is at least 95% identical to SEQ ID NO: 4 and the light chain variable region is at least 95% identical to SEQ ID NO: 5. In some embodiments, a total of 1 to 10 amino acids have been substituted, inserted and/or deleted in SEQ ID NO: 4. The substitutions, insertions, or deletions may occur in regions outside the CDRs (i.e., in the FRs). In the method of some embodiments, a total of 1 to 10, 1 to 5, or 1 to 3 amino acids have been substituted, inserted and/or deleted in SEQ ID NO: 5. The substitutions, insertions, or deletions may occur in regions outside the CDRs (i.e., in the FRs). In some embodiments, a total of 1 to 10, 1 to 5, or 1 to 3 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 10 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 5 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 3 amino acids have been substituted, inserted and/or deleted in SEQ ID NO: 4. The substitutions, insertions, or deletions may occur in regions outside the CDRs (i.e., in the FRs). In some embodiments, a total of 1 to 10, 1 to 5, or 1 to 3 amino acids have been substituted, inserted and/or deleted in SEQ ID NO: 4. Any of the anti-FGFR2 antibodies described herein may be afucosylated. For example, the antibody may be an IgG1 or IgG3 antibody that lacks fucose at Asn297.

Additional examples of anti-FGFR2b antibodies are the HuGAL-FR21, GAL-FR22 and GAL-FR23 antibodies described in U.S. Pat. No. 8,101,723 B2, incorporated by reference in its entirety herein. FIGS. 13 and 14 of U.S. Pat. No. 8,101,723 B2 show the amino acid sequences of the variable regions and full-length mature antibody chains of HuGAL-FR21, and are incorporated by reference herein. The heavy chain variable region sequences of antibody HuGAL-FR21, are underlined in FIG. 13 of U.S. Pat. No. 8,101,723 B2, and are specifically incorporated by reference herein. The light and heavy chain variable regions of GAL-FR22, for example, are provided as SEQ ID NOs: 7 and 8 in U.S. Pat. No. 8,101,723 B2, while the Kabat CDRs and the light and heavy chain variable regions are also provided in FIG. 16 of that patent, which are incorporated by reference herein. The GAL-FR21, GAL-FR22 and GAL-FR23 producing hybridomas are deposited at the American Type Culture Collection, PO Box 1549, Manassas VA, USA, 20108, as ATCC Numbers 9586, 9587, and 9408, on Nov. 6, Nov. 6, and Aug. 12, 2008, respectively. Thus, in some embodiments, the FGFR2 antibody is an antibody comprising the amino acid sequence of an antibody obtained from one of those three hybridoma strains.

Bemarituzumab is an afucosylated humanized monoclonal antibody that targets the fibroblast growth factor (FGF) receptor isoform 2b (FGFR2b) with a dual mechanism of FGF binding inhibition and antibody-dependent cellular cytotoxicity. The anti-FGFR2b antibody of any of the methods described herein may be bemarituzumab. Bemarituzumab comprises the heavy chain of SEQ ID NO: 2 and the light chain of SEQ ID NO: 3. In the method of some embodiments, the anti-FGFR2 antibody comprises the heavy chain of SEQ ID NO: 2 and the light chain of SEQ ID NO: 3, and is afucosylated. In the method of some embodiments, the anti-FGFR2b antibody is bemarituzumab. In the method of some embodiments, the anti-FGFR2b antibody comprises HCDR1-3 and LCDR1-3 of bemarituzumab. Bemarituzumab may be produced in a Chinese hamster ovary cell line that lacks the FUT8 gene, so that the produced antibody is glycosylated but lacks a core fucose in the polysaccharide portion of the antibody. The absence of the core fucose results in higher affinity for the Fc receptor FcγRIIIa compared to the fucosylated molecule and potentially enhances immune cell-mediated tumor cell killing.

Bemarituzumab inhibits FGF ligand-stimulated FGFR2b phosphorylation and cell proliferation in cell culture in FGFR2b overexpressing gastric, breast, and non-small cell lung cancer cell lines. Bemarituzumab also inhibits tumor growth in FGFR2b overexpressing gastric and breast xenograft models. Without being limited by theory, it is contemplated that mechanisms of action of bemarituzumab may include blocking ligand binding and downstream signaling, decreasing expression of the FGFR2b driver protein, and/or enhancing ADCC. Furthermore, without being limited by theory, it is contemplated that since bemarituzumab is specific for the FGFR2b receptor, it does not interfere with signaling of the other FGFs/FGFRs, including FGFR2c. In contrast to the FGFR tyrosine kinase inhibitors (TKIs), bemarituzumab does not inhibit FGF23 signaling. FGF23 is a ligand involved in calcium/phosphate metabolism and therefore, treatment with bemarituzumab is not associated with the hyperphosphatemia associated with the FGFR TKIs (Catenacci et al, 2020; Dienstmann et al, 2014; Sequist et al, 2014; Andre et al, 2013; Brown et al, 2005).

Bemarituzumab monotherapy has been investigated in a phase 1 dose-finding study (FPA144-001) and in combination with mFOLFOX6 chemotherapy in FGFR2b-positive gastric cancer in the FIGHT study. Bemarituzumab efficacy correlated with the degree of FGFR2b overexpression by immunohistochemistry (IHC) in gastric cancer and has demonstrated a manageable safety profile in combination with mFOLFOX6. Genomic and IHC data suggest that other carcinomas including may also have a significant rate of FGFR2b overexpression. Bemarituzumab blocks FGFR2b phosphorylation, downregulates the receptor, and inhibits downstream signaling. The effect on downstream signaling was measured by examining phosphorylation of a protein that is directly phosphorylated by the FGFR2 protein, FGFR substrate-2 (FRS2). Each of these mechanisms has been explored in vitro and in vivo and, without being limited by theory, appears to contribute to the anti-tumor activity of bemarituzumab. In FGFR2b-overexpressing human tumor xenograft models, bemarituzumab shows dose-related anti-tumor activity with regression and complete responses at well-tolerated doses.

Bemarituzumab demonstrated consistent pharmacokinetic (PK) behavior following intravenous (IV) administration in rats and cynomolgus monkeys, and the PK characteristics observed were consistent across all studies. The half-life was dose-dependent, ranging from 0.8 days at the lowest doses (1 to 1.5 mg/kg) to at least 8 days at the highest doses (100 to 150 mg/kg) tested in cynomolgus monkeys. Bemarituzumab demonstrated dose-dependent, nonlinear PK that was marked by a faster clearance at the terminal phase of the plasma concentration time profile and a greater than dose proportional increase in exposure (area under the concentration time curve [AUC]) with increasing dose. Target-mediated clearance was saturable, marked by dose-proportional increases in exposure at doses exceeding this level when dosed at weekly intervals. The PK studies supporting the toxicokinetic studies showed dose-dependent increases in exposure (AUCs) supporting the reliability of these studies to assess toxicity. Significant reproductive and developmental toxicities were observed at all dose levels (5 to 100 mg/kg/doses) in the embryo-fetal development with prenatal and postnatal development study. As such, it is contemplated that in some embodiments, subjects treated with bemarituzumab are not pregnant.

Bemarituzumab has demonstrated an acceptable safety profile. Identified risks when used in combination with mFOLFOX6 include corneal toxicity, infusion related reactions, gastrointestinal toxicity (stomatitis and mucosal inflammation), nail toxicity and increase in AST and ALT. Corneal events are very common with bemarituzumab with the most common adverse event being dry eye. Although nearly all of the events have been non-serious, grade 3 events such ulcerative keratitis and punctate keratitis which can lead to decreases in visual acuity have been observed. The majority of the corneal events typically resolve with treatment interruption or discontinuation and standard of care interventions for the corneal events. As such, it is contemplated that in some embodiments, subjects treated with bemarituzumab are further treated with ocular lubricants. The ocular lubricants may be administered prophylactically to reduce the risk of corneal events.

In some methods described herein, bemarituzumab may be provided in a drug product composition comprising or consisting essentially of an aqueous solution comprising 20 mg/mL bemarituzumab, L-histidine, sucrose, and polysorbate 20 at pH 6.0. For example, the solution may comprise or consist essentially of or consist of 20 mg/mL bemarituzumab, 20 mM L-histidine, 270 nM sucrose, and 0.01% (w/v) polysorbate 20 at pH 6.0

The anti-FGFR2b antibody, such as bemarituzumab, may be administered intravenously in methods described herein.

Methods of Treating Solid Tumors with FGFR2b Overexpression

### Solid Tumors

Methods of treating solid tumors in a subject, such as squamous cancer, ER− PR− HER2/neu− (“triple-negative”) breast cancer, intrahepatic cholangiocarcinoma, lung adenocarcinoma, and gynecological malignancy, are described herein. The squamous cancer may be, for example, head and neck cancer. The gynecological malignancy may be, for example, ovarian epithelial cancer (including fallopian tube cancer and primary peritoneal cancer), endometrial cancer, or cervical cancer. In some embodiments, the solid tumor is selected from squamous cancer, triple-negative breast cancer, pancreatic ductal adenocarcinoma, intrahepatic cholangiocarcinoma, colorectal adenocarcinoma, and gynecological malignancy.

In some embodiments, the anti-FGFR2b antibody monotherapy is administered as a second line or beyond therapy for the solid tumor, such as third line or beyond. The term “second line therapy,” as used herein, refers to treatment for a disease or condition after the initial treatment (“first line” treatment or therapy) has failed, stopped working, or has side effects that are not tolerated by the patient. Therapies or treatments administered after the second line therapy may be referred to as “beyond” second line therapy, or numerically (e.g., “third” or “fourth” line therapy). In some embodiments, the anti-FGFR2b antibody monotherapy is administered as a second line or beyond therapy following a first line therapy that comprises, for example, chemotherapy, radiation, and/or immunotherapy. For example, the solid tumor may be post platinum-based chemotherapy, post-PD-1 inhibitor therapy, post-poly (ADP-ribose) polymerase inhibitor (PARPi) therapy (if BRCA-mutated), post-anti-trop-2 therapy, or post-targeted therapy. In some embodiments, the solid tumor may be a squamous cancer that is post platinum-based chemotherapy and/or post-PD-1 inhibitor. In some embodiments, the solid tumor is a triple negative breast cancer that is post chemotherapy, post-PARPi (if BRCA-mutated), post-PD-1 inhibitor therapy, and/or post-anti-trop-2 therapy. In some embodiments, the solid tumor may be a pancreatic ductal adenocarcinoma that is post-platinum based chemotherapy. In some embodiments, the solid tumor may be an intrahepatic cholangiocarcinoma that is post-platinum based chemotherapy and post-targeted therapy, if eligible for targeted therapy. In some embodiments, the solid tumor may be a colorectal adenocarcinoma that is post-bevacizumab therapy, post-oxaliplatin-based chemotherapy, post-irinotecan-based chemotherapy, and/or post-additional prior therapy based on RAS, BRAF, and dMMR/MSI-H status. In some embodiments, the solid tumor may be a gynecological malignancy that is post platinum-based chemotherapy and/or is platinum chemotherapy resistant.

### Methods Comprising Administering an Anti-FGFR2b Antibody

The methods described herein may comprise administering an anti-FGFR2b antibody monotherapy. For example, the anti-FGFR2b antibody monotherapy may comprise an every two weeks (Q2W) regimen of a first administration of the anti-FGFR2b antibody at a dose of greater than 20 mg/kg to no more than 30 mg/kg, followed two weeks after the first administration and Q2W thereafter by subsequent administrations of the anti-FGFR2b antibody each at a dose of 12-20 mg/kg, wherein the subsequent administrations are at a lower dose than the first administration. In another example, the anti-FGFR2b antibody monotherapy may comprise an every two weeks (Q2W) regimen of the anti-FGFR2b antibody at a dose of greater than 10 mg/kg to no more than 20 mg/kg, and one week after the first administration of the anti-FGFR2b antibody, administering a single subsequent administration of the anti-FGFR2b antibody at a dose of 5-10 mg/kg. A subsequent administration or dose of the anti-FGFR2b antibody may be referred to as an “intervening” dose.

In some of the methods, the anti-FGFR2b antibody is administered to the subject Q2W at a dose of greater than 20 mg/kg to no more than 30 mg/kg, such as greater than 20 mg/kg to no more than 25 mg/kg. In some of the methods, the anti-FGFR2b antibody is administered to the subject Q2W at a dose of about 22-25 mg/kg (e.g., 22 mg/kg, 23 mg/kg, 24 mg/kg, 25 mg/kg, or a range defined by any two of the foregoing values). In some of the methods, the anti-FGFR2b antibody is administered to the subject Q2W at a dose of 22 mg/kg. In accordance with some methods described herein, the additional dose or “intervening” dose of the anti-FGFR2b antibody may be 12-20 mg/kg, such as 12-17 mg/kg, and may be administered two weeks after the first administration of the anti-FGFR2b antibody and Q2W thereafter. In accordance with some methods described herein, the first administration of the anti-FGFR2b antibody may be followed two weeks Q2W thereafter by subsequent administrations of the anti-FGFR2b antibody each at a dose of about 12 mg/kg, about 13 mg/kg, about 14 mg/kg, about 15 mg/kg, about 16 mg/kg, about 17 mg/kg, or a range defined by any two of the foregoing values. By way of example, the additional dose or “intervening” dose may be about 15 mg/kg.

In some of the methods, the anti-FGFR2b antibody is administered to the subject Q2W at a dose of greater than 10 mg/kg to no more than 20 mg/kg. In some of the methods, the anti-FGFR2b antibody is administered to the subject Q2W at a dose of about 12-17 mg/kg (e.g., 12 mg/kg, 13 mg/kg, 14 mg/kg, 15 mg/kg, 16 mg/kg, 17 mg/kg, or a range defined by any two of the foregoing values). In some of the methods, the anti-FGFR2b antibody is administered to the subject Q2W at a dose of 15 mg/kg. In some embodiments, one week after the first administration of the anti-FGFR2b antibody, the method may comprise administering a single subsequent administration of the anti-FGFR2b antibody at a dose of 5-10 mg/kg (e.g., 5 mg/kg, 6 mg/kg, 7 mg/kg, 8 mg/kg, 9 mg/kg, 10 mg/kg, or a range defined by any two of the foregoing values). By way of example, the additional dose or “intervening” dose may be about 7-8 mg/kg, such as about 7.5 mg/kg.

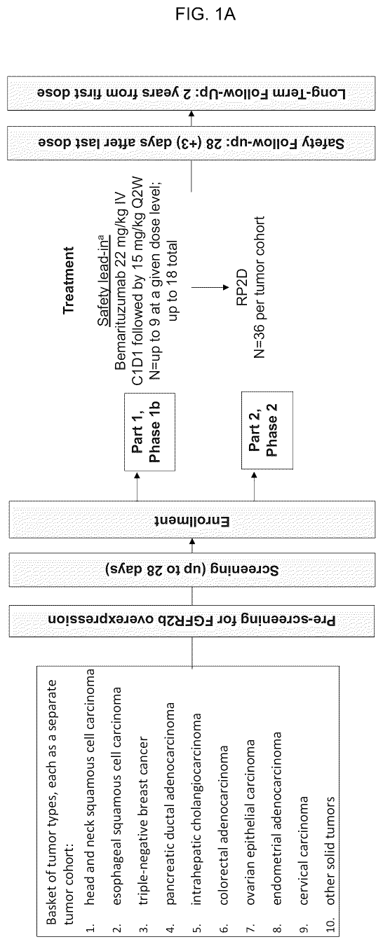

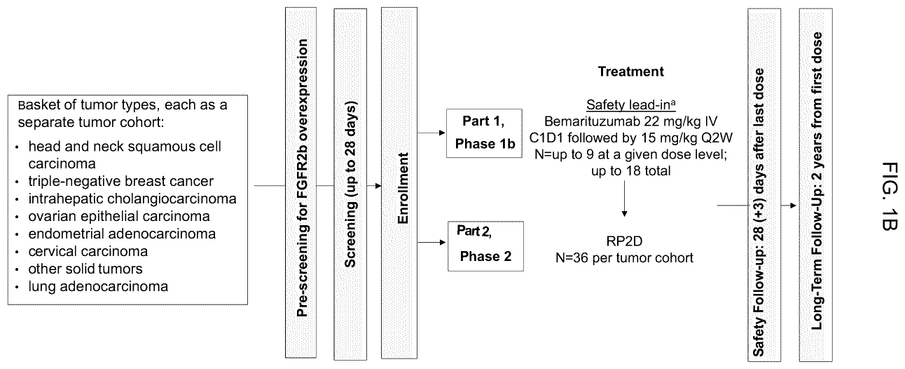

For any of the methods described herein, the anti-FGFR2b antibody may be bemarituzumab. Methods of treating solid tumors in a subject according to some embodiments are depicted in FIG. 1.

Described in accordance with methods of some embodiments is a study to evaluate the safety and efficacy of bemarituzumab monotherapy in subjects across multiple solid tumors with FGFR2b overexpression and relapsed/refractory unresectable and/or metastatic disease. The study may comprise a dosing and scheduling of bemarituzumab as described in Example 2.

For any of the methods of treating solid tumors described herein, cells of the solid tumor of the subject may express FGFR2b. For example, cell of the solid tumor of the subject may overexpress FGFR2b protein, overexpress FGFR2b mRNA, or comprise an FGFR2b gene amplification. In some methods, cells of the solid tumor express FGFR2b protein as determined by immunohistochemistry (IHC). For example, at least 5% (e.g., 5%, 10%, or 20%) of the cells of the subject's solid tumor may be positive for FGFR2b as determined by IHC. For example, the cells of the subject's solid tumor may have an FGFR2b staining intensity of 2+ and/or 3+. For example, at least 5% (e.g., at least 5%, 10%, or 20%) of the solid tumor cells may have an FGFR2b staining intensity of 1+, 2+ or 3+. If the cells of the subject's solid tumor may have an FGFR2b staining intensity of 2+ or 3+, or if at least 5% (e.g., at least 5%, 10%, or 20%) of the solid tumor cells may have an FGFR2b staining intensity of 1+, 2+ or 3+, the solid tumor of the subject may be considered to overexpress FGFR2b. For example, at least 5% of the cells of the subject's solid tumor may have an FGFR2b staining intensity of 2+ and/or 3+. For example, at least 10% of the cells of the subject's solid tumor may have an FGFR2b staining intensity of 2+ and/or 3+. It is contemplated that subjects having solid tumor that overexpress FGFR2b are especially likely to benefit from methods of treatment comprising administering anti-FGFR2b antibodies (such as bemarituzumab) described herein. Optionally, cells of the solid tumor are also assessed for PD-L1 expression, for example by IHC.

The following examples further illustrate the invention but, of course, should not be construed as in any way limiting its scope.

### Example 1: Administration of Bemarituzumab to Breast Cancer and Lung Cancer Cell Lines

ADCC activity of bemarituzumab was measured in vitro in squamous lung cancer cells lines and breast cancer cell lines that were determined by flow cytometry to exhibit surface expression of FGFR2b. Squamous lung cancer cell lines (KNS-62, LC1F, HARA, EPLC-272H, SW900, NCIH2170, LUDLU1, and SW1573) and triple negative breast cancer (TNBC) cell lines (HCC1569, HCC1806, HCC38, HCC1187, BT20, and HCC1937) were evaluated for FGFR2b mRNA expression [ENST00000457416.6 [FPKQ]] and for surface expression of the FGFR2b protein by flow cytometry. As a positive control, gastric cancer cell lines SNU16-Luc, SNU16, and KATOIII were also evaluated. In the flow cytometry experiments, cells were incubated with bemarituzumab or a control antibody of the same isotype, and then binding was detected using an anti-human IgG1 antibody conjugated to allophycocyanin (APC). Mean fluorescence intensity (MFI) was quantified by flow cytometry. As shown in Table 1 below, squamous lung cancer cell lines KNS-62, EPLC-272H, LC1F, HARA, SW900, and LUDLU1 had an MFI of at least 10, as did triple negative breast cancer (TNBC) cell lines HCC1569, HCC1806, HCC38, BT20, and HCC1937.

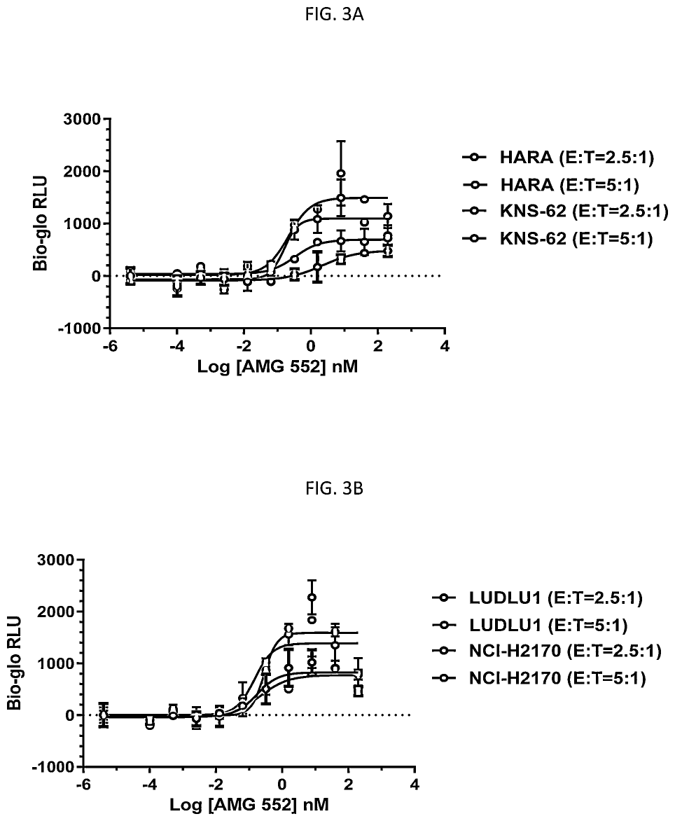

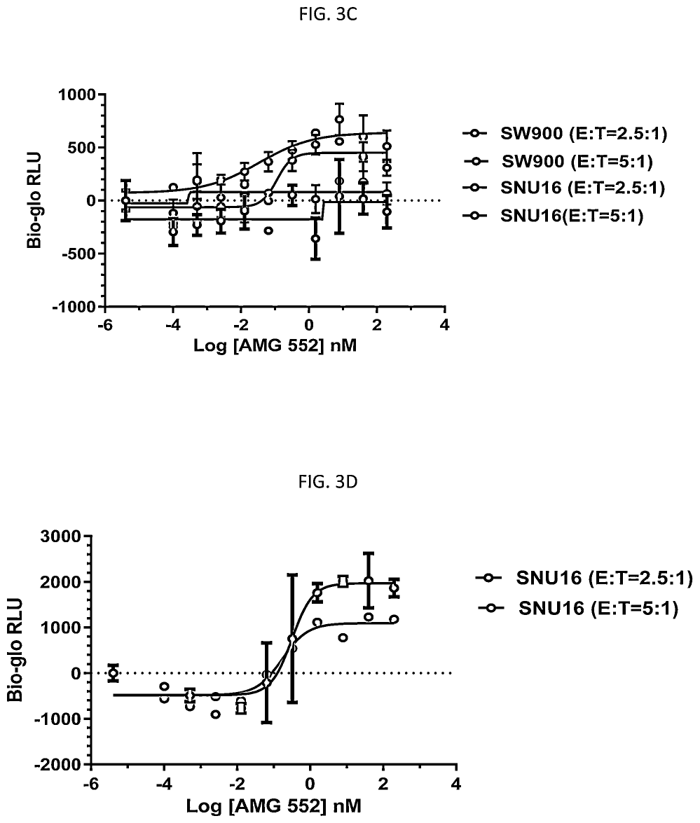

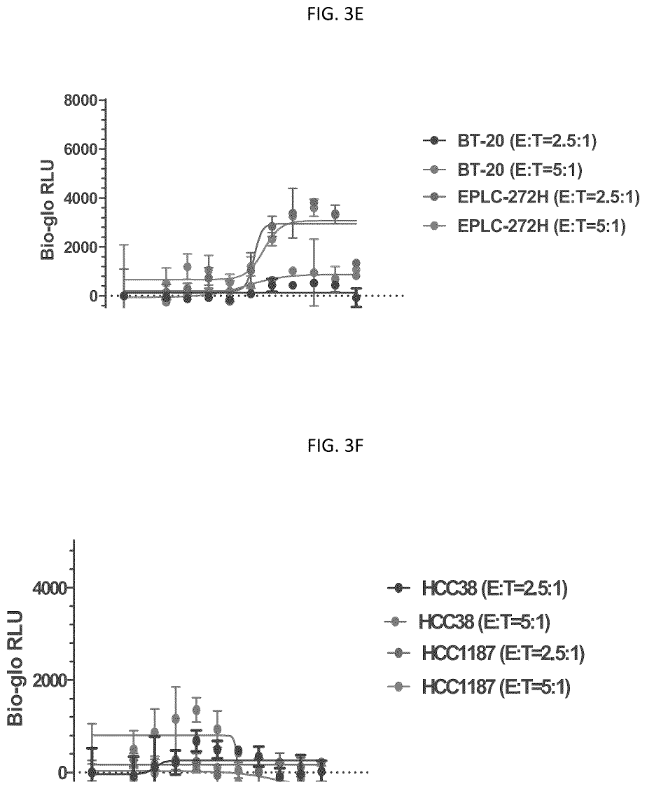

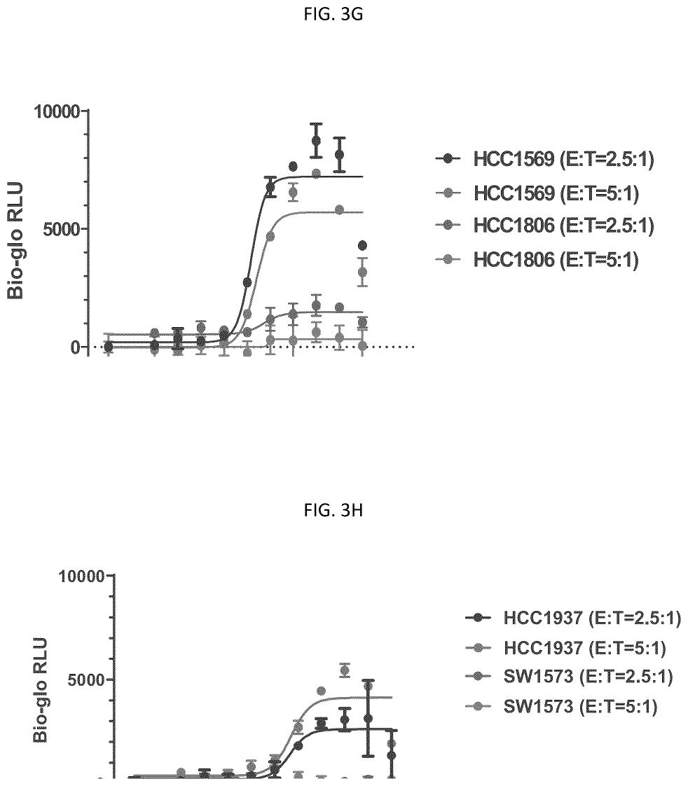

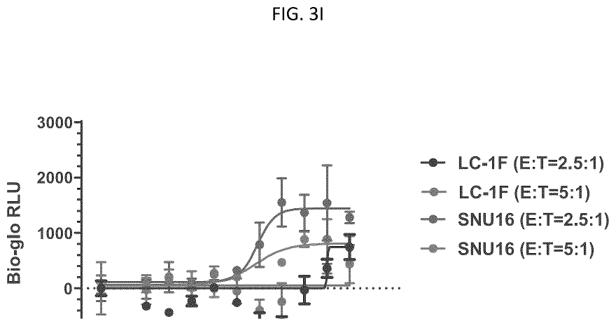

Each of the cell lines was evaluated for effects of bemarituzumab on antibody-dependent cellular cytotoxicity (ADCC). Cancer cell lines were co-cultured with Jurkat-Luc effector cells in a 96 well plate, with fixed effector cell number (75,000 cells per well) and a 2.5:1 or 5:1 effector to target cell ratio. Cells were treated with bemarituzumab at 20 mg/mL and then were incubated for 20 hours at 37° C. ADCC was assessed using a ADCC Reporter Bioassay from Promega (G7018), which uses a luminescent readout. ADCC curves are summarized in FIGS. 3A-3I. ADCC activity was observed in squamous lung cancer cell lines and breast cancer cell lines that exhibited surface expression of FGFR2b above 10 MFI. Thus, it can be concluded that bemarituzumab is capable of inducing ADCC in FGFR2b-positive triple negative breast cancer cells and squamous lung cancer cells.

TABLE 1

| | | | Mean | |
|---|---|---|---|---|
| | | FGFR2b mRNA | Fluorescence | |
| Tumor | | (ENST00000457416.6) | Intensity by | ADCC |
| Type | Cell Line | [FPKQ] | FACS | activity |
| Squamous | KNS-62 | 8.9 | 572 | ++ |
| Lung | EPLC-272H | 7.9 | 759.2 | ++ |
| Cancer | LC1F | 3.2 | 123 | − |
| | HARA | 3.2 | 401.1 | + |
| | SW900 | 1.6 | 10.3 | − |
| | NCIH2170 | 1.1 | 152.9 | ++ |
| | LUDLU1 | 0.9 | 638.5 | + |
| | SW1573 | 0.1 | −1.2 | − |
| TNBC | HCC1569 | 8.9 | 1365 | +++ |
| | HCC1806 | 6.6 | 401 | + |
| | HCC38 | 4.5 | 346 | − |
| | HCC1187 | 2.4 | −6 | − |
| | BT20 | 1.9 | 252.5 | +/− |
| | HCC1937 | 1.9 | 689.9 | ++ |
| | SNU16 | 548.3 | 7799.3 | ++ |
| Gastric | SNU16-Luc | 548.3 | 7962.5 | ++ |
| Cancer | KATOIII | 410.4 | 20375.2 | Not |
| | | | | tested |

### Example 2: A Phase 1b/2 Basket Study of Bemarituzumab Monotherapy in Solid Tumors with FGFR2b Overexpression

This example describes a phase 1b/2 open-label, multicenter exploratory, signal finding basket study to evaluate the efficacy and safety of bemarituzumab monotherapy in subjects across multiple solid tumors with FGFR2b overexpression and relapsed/refractory unresectable and/or metastatic disease.

The study includes a pre-screening period to demonstrate FGFR2b overexpression via central testing, a 28 day screening period, a treatment period, a safety follow-up (SFU) period, and a long term follow-up (LTFU) period. Subjects who discontinue bemarituzumab will undergo a SFU visit 28 (+3) days after the last dose of study treatment. In addition, subjects will undergo LTFU for survival approximately every 3 months (±1 month) after the SFU visit for up to 2 years from the first dose of bemarituzumab. Subjects will receive treatment until disease progression, unacceptable toxicity, subject request, or death (whichever occurs first).

Radiographic assessment will be performed by the investigator according to Response Evaluation Criteria in Solid Tumors (RECIST) v1.1 and will be performed every 8 weeks (±7 days) from cycle 1 day 1 until week 56 and then every 12 weeks (±14 days) until radiographic progression or initiation of subsequent anti-cancer therapy. Primary and secondary objectives and endpoints are summarized in Table 2 below.

TABLE 2

| | |
|---|---|
| Objectives | Endpoints |

Phase 1b - Safety Lead-in
Primary

| | |
|---|---|
| To observe the safety | Dose-limiting toxicities (DLTs), |
| and tolerability | treatment-emergent adverse events, |
| of bemarituzumab | treatment-related adverse events, and |
| | clinically significant changes in vital |
| | signs, visual acuity, and clinical |
| | laboratory tests |

Secondary

| | |
|---|---|
| To evaluate preliminary | Objective response (OR) |
| antitumor activity | (OR = complete response [CR] + |
| | partial response [PR]), measured by |
| | computed tomography (CT) or |
| | magnetic resonance imaging (MRI) as |
| | determined by investigator per |
| | Response Evaluation Criteria in Solid |
| | Tumors version 1.1 (RECIST v1.1) |
| | Disease control (DC) (CR, PR, or |
| | stable disease [SD]) |
| | Duration of response (DOR), defined |
| | as the time from first documentation |
| | of objective response (as determined |
| | by investigator per RECIST v1.1) |
| | until the first documentation of |
| | disease progression or death due to |
| | any cause, whichever occurs first. |
| | Only subjects who have achieved |
| | objective response will be evaluated |
| | for DOR. Duration of response will |
| | be censored at the last evaluable |
| | post-baseline tumor assessment prior |
| | to subsequent anticancer therapy. |
| | Time to response (TTR) |
| | Progression-free survival (PFS), |
| | defined as time from first dose of |
| | investigational product until the first |
| | documentation of radiologic disease |
| | progression or death due to any cause. |
| | Progression-free survival will be |
| | censored at the last evaluable post- |
| | baseline tumor assessment prior to |
| | subsequent therapy. Progression will |
| | be based on RECIST v1.1 (derived |
| | utilizing investigator tumor |
| | assessments) |
| | Overall survival (OS), defined as time |
| | from first dose of investigational |
| | product until death from any cause. |
| | Subjects still alive will be censored at |
| | the date last known to be alive |
| Characterize the | PK parameters for bemarituzumab |
| pharmacokinetics | including, but not limited to, area |
| (PK) of bemarituzumab | under the concentration time curve |
| | (AUC), maximum observed serum |
| | concentration (Cₘₐₓ), and the |
| | observed concentration at the end of a |
| | dose interval (C_{trough}) |

Phase 2
Primary

| | |
|---|---|
| To evaluate preliminary | OR |
| antitumor activity | |

Secondary

| | |
|---|---|
| To evaluate other | DC |
| measures of preliminary | DOR |
| antitumor activity | TTR |
| | PFS |
| | OS |
| To evaluate the | Treatment-emergent adverse events, |
| safety and tolerability | treatment-related adverse events, and |
| of bemarituzumab | clinically significant changes in vital |
| | signs, visual acuity, and clinical |
| | laboratory tests |
| Characterize the PK of | PK parameters for bemarituzumab |
| bemarituzumab monotherapy | including, but not limited to, AUC, |
| | Cₘₐₓ, and C_{trough} |

The study includes 2 parts: monotherapy dose exploration (Part 1, phase 1b) across tumor types (i.e., 9 to 18 subjects in total regardless of tumor type), followed by monotherapy dose expansion (Part 2, phase 2) for each of the 8 tumor cohorts below at the selected dose level from Part 1. For both parts of the study, tumor samples from subjects are required to demonstrate FGFR2b overexpression prior to entering this study. A tumor specimen collected since completion of the most recent cancer therapy is recommended.

This study will enroll subjects into 8 tumor cohorts according to cancer type:
1. head and neck squamous cell carcinoma
2. triple-negative breast cancer (ER−, PR−, HER2/neu−)
3. intrahepatic cholangiocarcinoma
4. lung adenocarcinoma
5. ovarian epithelial carcinoma, including fallopian tube cancers and primary peritoneal cancers
6. endometrial adenocarcinoma
7. cervical carcinoma
8. other solid tumors

The requirements for number of previous lines of therapy differ according to study cohort as outlined in the inclusion criteria below.

The study may be amended to add or remove cohorts based on enrollment rate, prevalence of FGFR2b overexpression in pre-screening, preliminary efficacy and safety data, and evolving data on other potential solid tumor indications with FGFR2b overexpression. Study cohorts may be modified to specific histologic subtypes based on emerging data on FGFR2b overexpression and response rates. Recruitment/enrollment in any of the above cohorts may present some challenges due to the low possible frequency of FGFR2b overexpression in the specific disease settings. Therefore, the Sponsor reserves the right to stop recruitment/enrollment of any of the cohorts due to slow or absent accrual. Cohort 10 (other solid tumors) will be closed to enrollment when all other cohorts are closed, regardless of the number of patients recruited at that time.

Part 1 begins with Dose Level 1 (22 mg/kg intravenous [IV]cycle 1 day 1 followed by 15 mg/kg IV every 2 weeks [Q2W] starting on day 15). The study DLT evaluation period is 28 days. Once 3 to 6 subjects have completed the DLT evaluation period, a Dose Level Review Team (DLRT) meeting will be convened. Depending on the observed safety data, the following may occur: (1) additional enrollment to Dose Level 1; or (2) dose de-escalation to Dose Level 1A; or (3) initiation of Part 2 of the study. A Modified Toxicity Probability Internal (mTPI)-2 design using a target toxicity probability of 0.25 with an acceptable toxicity probability interval of (0.20, 0.30) will be used to derive escalation/de-escalation guidelines. Part 1 will continue until a maximum sample size of 18 is reached or the number of subjects treated at a given dose level reaches 9, and the mTPI-2 algorithm instructs to stay at that dose level. In addition, 3 to 6 Japanese subjects will be enrolled, either as part of the initial dose evaluation in Part 1 or as backfill if a recommended phase 2 dose (RP2D) is determined. Japanese subjects may enroll in Part 2 once the DLRT has deemed the global RP2D safe for Japanese subjects.

### Number of Subjects

A total of between 288 to 303 subjects will be enrolled in the study, with 9 to 18 subjects in Part 1, irrespective of tumor cohort, and potentially up to 6 additional Japanese subjects. In Part 2, up to 36 subjects in each of 10 planned tumor cohorts will be enrolled (subjects in Part 1 assigned to the same dose level used in Part 2 will contribute to the 36-subject total so some tumor cohorts may enroll less than 36 subjects in Part 2).

### Summary of Subject Eligibility Criteria

Subjects are ≥18 years of age (or legal adult age within country, whichever is older) with histologically or cytologically confirmed cancer of the types outlined in the study design. Subjects must have unresectable, locally advanced, or metastatic disease. Subjects must have FGFR2b overexpression as determined by centrally performed immunohistochemistry (IHC) testing. All subjects must have measurable disease per RECIST v.1.1.

### Treatments

The planned dose of bemarituzumab (Dose Level 1) in this study is 22 mg/kg IV cycle 1 day 1 followed by 15 mg/kg IV Q2W thereafter starting on day 15. One cycle of treatment will be 14 days. Dose Level 1A, including bemarituzumab 15 mg/kg IV Q2W starting cycle 1 day 1 plus 1 additional 7.5 mg/kg dose on cycle 1 day 8 only, may be explored if dose de-escalation is required from Dose Level 1.

### Statistical Considerations

### Sample Size Considerations

In Part 1, there is a 92% probability of observing at least 1 DLT if the true DLT rate is 25% with 9 subjects treated at a dose level. In Part 2, the Clopper-Pearson Exact 95% lower confidence limits corresponding to observed objective response rate (ORRs) of 11.1%, 16.7%, 22.2%, 27.8%, and 33.3% with 36 subjects are 3.1%, 6.4%, 10.1%, 14.2%, and 18.6%, respectively; subjects in Part 1 assigned to the same dose level used in Part 2 will contribute to the 36 subject total.

### Interim Analyses

In Part 1, the DLRT will convene to review all available safety, tolerability, laboratory, and PK data during and after Part 1 is completed (28 days following last subject enrolled in Part 1).

In Part 2, a data review team (DRT), will review safety data after a specified number of subjects in the full analysis set, regardless of tumor type, have had the opportunity to be followed for 8 weeks. To make their assessment, the DRT will use their clinical judgement when reviewing all relevant safety data and use the stopping guidelines which are based on having a >85% Bayesian posterior probability that the posterior probability of the grade 4+ treatment-related adverse event rate exceeds 20% using a beta (1,1) prior distribution.

In addition to the safety data review, the DRT will oversee non-binding interim analyses for futility that are planned to occur after the first 12 and 24 subjects dosed in a given tumor cohort have had the opportunity to complete the 16-week disease assessment (two scans). Enrollment will not be paused in order to conduct the futility analyses. Stopping for futility will be based on having a <20% predictive probability that the ORR will be >15% after all 36 subjects are enrolled and have the opportunity to complete the 16-week disease assessment. A noninformative beta (1, 1) prior distribution will be used. A cohort may stop for futility if 0 out of 12 subjects or ≤1 out of 24 subjects have an OR. Before the primary analysis for the entire study, additional interim analyses will be performed by tumor cohort on select efficacy and safety endpoints once all subjects enrolled in that tumor cohort have had the opportunity to complete the 16-week disease assessment.

### Analysis Methods

The primary analysis of efficacy and safety will be based on all enrolled subjects who received at least one dose of investigational product. Continuous variables will be described with the mean, median, quartiles, minimum, and maximum. Categorical data will be summarized with frequency counts and percentages. Response rates will be summarized with exact 95% confidence intervals. Time-to-event endpoints will be summarized with Kaplan-Meier (KM) quartiles and rates at select timepoints. The final analysis will occur when all subjects across all tumor cohorts complete the study. The time-to-event endpoints will be updated with further follow-up at the final analysis. No statistical hypothesis is tested in this study.

### Rationale for Bemarituzumab Dose

For Phase 1 of this study, bemarituzumab at 22 mg/kg cycle 1 day 1 followed by 15 mg/kg Q2W starting on day 15 was selected based on the following data.

In the phase 1 FPA144-001 monotherapy dose escalation study, bemarituzumab demonstrated linear clearance from 1 mg/kg to 15 mg/kg in subjects with solid tumors including gastric cancers. In the linear dose range, maximum observed serum concentration (Cₘₐₓ) and AUC increased dose proportionally. The estimated half-life by noncompartmental analysis ranged from 6.01 to 11.7 days across 1 mg/kg to 15 mg/kg, which supports Q2W or less frequent dosing.

The target observed concentration at the end of a dose interval (C_{trough}) for bemarituzumab of ≥60 g/mL was derived from nonclinical studies including binding affinity for human FGFR2b-Fc and human FcγRIIIa (V158), receptor occupancy in vitro, and an efficacy study in vivo.

Supporting the hypothesis that 60 g/mL should be the target minimum C_{trough}, all subjects who demonstrated a partial response (PR) in the FIH FPA144-001 trial with FGFR2b overexpression achieved the target C_{trough,ss} of ≥60 μg/mL, regardless of dose levels. Among all subjects treated with 15 mg/kg Q2W dosing in Study FPA144-001, 23 of 51 subjects achieved target C_{trough} concentration on day 15. At the same dose by the 8th week (steady state), 14 of 16 subjects achieved target C_{trough} of ≥60 μg/mL.

Since shortening the time to achieve target C_{trough} levels may help to maximize the potential benefit from bemarituzumab, 1 additional dose of 7.5 mg/kg on cycle 1 day 8 was added to achieve the goal.

The observed PK data from the phase 1 portion of Study FPA144-004 indicated that all subjects with PK data treated at 15 mg/kg Q2W with 1 additional dose of 7.5 mg/kg on cycle 1 day 8 achieved target C_{trough} concentration on day 15 and Cₘₐₓ was within the range observed in Study FPA144-001. Therefore, the addition of a single dose of 7.5 mg/kg on cycle 1 day 8 minimized the time to reach target C_{trough} without increasing Cₘₐₓ.

The FPA144-004 study with Q2W dosing of bemarituzumab at 15 mg/kg with an additional dose of 7.5 mg/kg dose on cycle 1 day 8 in combination with mFOLFOX6 achieved pre-specified statistical significance across all three of its prespecified efficacy endpoints including objective ORR, PFS, and OS in subjects with FGFR2b-positive, non HER2 positive frontline advanced for gastric cancer and gastroesophageal junction cancer. In addition, population PK analysis using PK data from Studies FPA144-001, FPA144-002, and FPA144-004 indicated that no covariate was expected to have clinically meaningful effects on bemarituzumab exposure in gastric cancer and gastroesophageal junction cancer population and treatment although baseline body weight, baseline albumin, gender, and combo therapy were identified as statistically significant covariates on the PK of bemarituzumab. No dose adjustment based on any of these factors is warranted. In addition, preliminary exposure-response analysis indicated that based on the manageable safety and favorable efficacy data, bemarituzumab 15 mg/kg Q2W with 1 additional dose of 7.5 mg/kg on cycle 1 day 8 adequately balances the safety and efficacy.

### Rationale for Loading Dose

In this study a loading dose of 22 mg/kg on day 1 of cycle 1 (ahead of 15 mg/kg Q2W from day 15 onwards) is added to achieve early target serum exposures for bemarituzumab in subjects. As described above, in the previous clinical study for bemarituzumab (FPA-144-004) an additional dose of 7.5 mg/kg on day 8 of cycle 1 was added after the 15 mg/kg dose on day 1 of cycle 1 to help achieve these early target exposures (target trough coverage). Population PK modeling and simulation indicates that a dose of 22 mg/kg on day 1 of cycle 1 achieves similar C_{trough} on day 15 as the additional dose of 7.5 mg/kg on day 8 of cycle 1 after the 15 mg/kg dose on day 1 of cycle 1. Hence, the loading dose of 22 mg/kg on day 1 not only helps achieve early target exposures but also improves subject convenience by removing the need for the additional day 8 dose of 7.5 mg/kg.

For Phase 2 of this study, the recommended dose of bemarituzumab will be identified based on an evaluation of the overall safety, tolerability, and PK of the Phase 1 of the study.

### End of Study

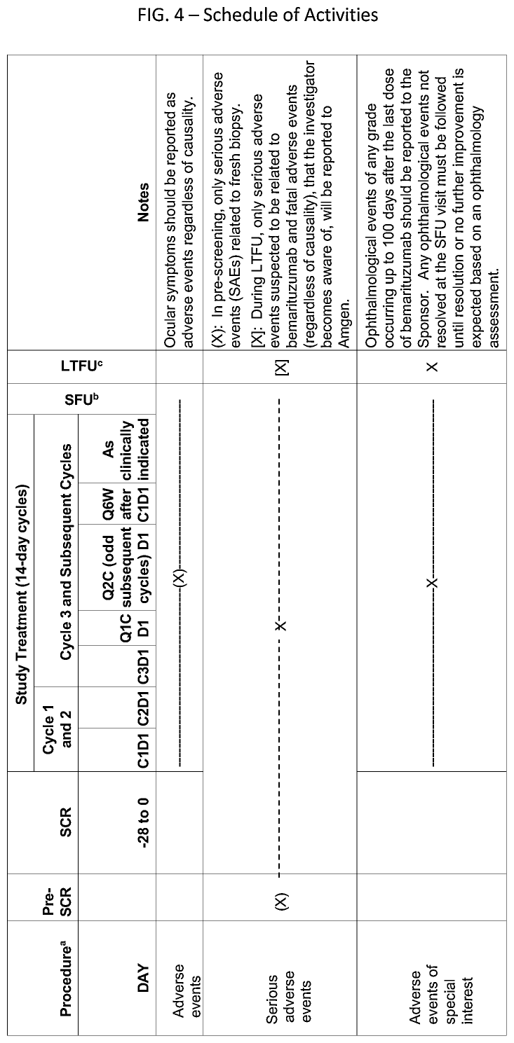

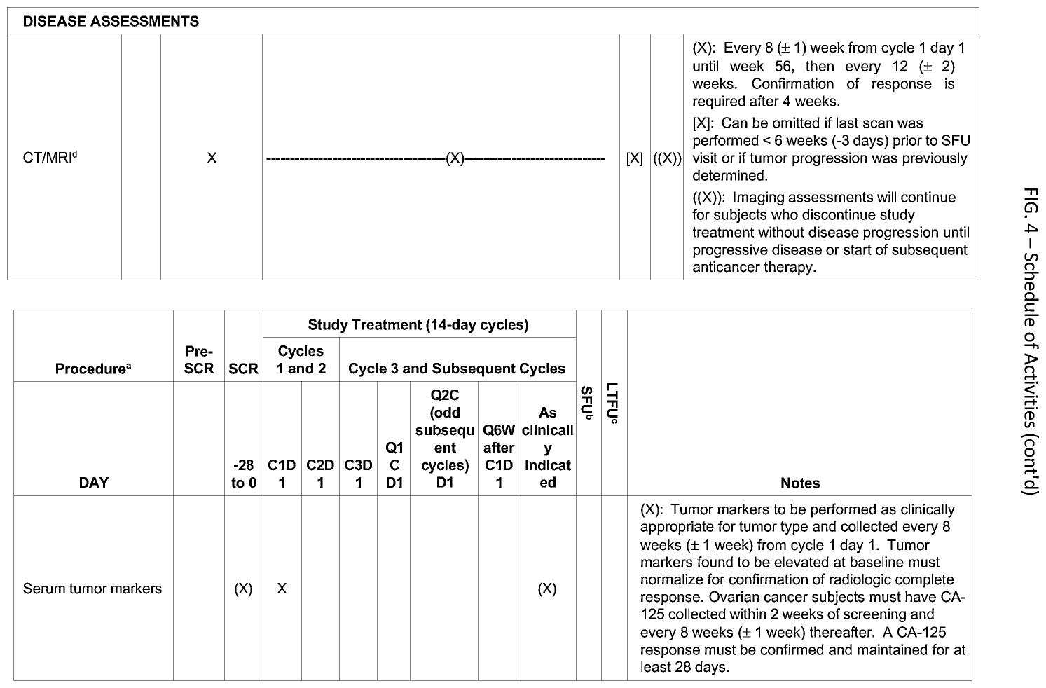

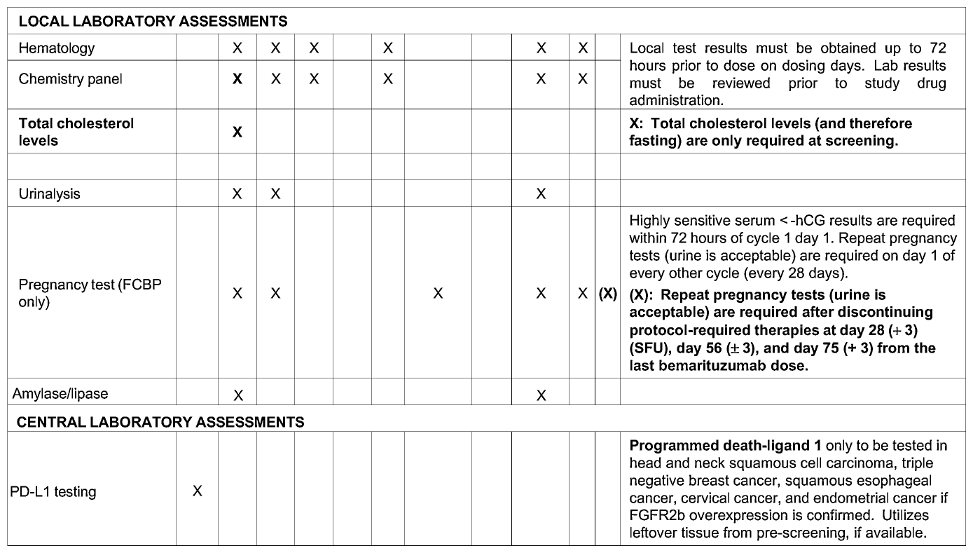

An individual subject is considered to have completed the study if he/she has completed the last visit shown in the Schedule of Activities detailed in FIG. 4.

The end of study date is defined as the date when the last subject across all sites is assessed or receives an intervention for evaluation in the study (i.e., last subject last visit), including any additional parts in the study (e.g., long-term follow-up, antibody testing), as applicable.

### Study Population

Investigators will maintain a pre-screening log and screening log of all potential study candidates that includes limited information about the potential candidate (e.g., date of screening). Eligibility criteria will be evaluated during screening. Before any study-specific activities/procedures, the appropriate written informed consent must be obtained. Prospective approval of protocol deviations to recruitment and enrollment criteria, also known as protocol waivers or exemptions will not be provided.

### Inclusion Criteria

Subjects are eligible to be included in the study only if all of the following criteria apply.
Subject has provided informed consent/assent prior to initiation of any study specific activities/procedures.
Age ≥18 years (or legal adult age within country, whichever is older) at the time that the Informed Consent Form (ICF) is signed.
Histologically or cytologically confirmed cancer of one of the following types, refractory to or relapsed after at least 1 prior standard therapeutic regimen in the advanced/metastatic setting, as specified below. If no standard of care therapies exist for the subject, or the subject cannot tolerate or refuses standard of care anticancer therapy, the subject may be allowed to participate on the study after discussion between the investigator and Amgen medical monitor. Subjects who have not received all approved or standard treatments for their cancer must be informed that these alternatives to receiving bemarituzumab are available prior to consenting to participate in the trial.
head and neck squamous cell carcinoma: 1 line of therapy
triple-negative breast cancer: 2 lines of therapy
Intrahepatic cholangiocarcinoma 1 line of therapy
lung adenocarcinoma: at least platinum-based chemotherapy, checkpoint inhibitor, and targeted therapy (i.e., if molecular testing has identified targetable mutations in EGFR, ALK, etc.)
platinum-resistant ovarian epithelial carcinoma, including fallopian tube cancers and primary peritoneal cancers, defined as progression during or within 6 months of a
platinum containing regimen: 1 line of therapy
endometrial adenocarcinoma: 1 line of therapy
cervical carcinoma: 1 line of therapy
other solid tumors: 1 line of therapy
Disease that is unresectable, locally advanced, or metastatic (not amenable to curative therapy)
Tumor overexpresses FGFR2b as determined by centrally performed immunohistochemistry (IHC) testing
Measurable disease per RECIST v1.1
Eastern Cooperative Oncology Group (ECOG) performance status of 0 or 1
Adequate hematologic and organ function, defined as follows:
   Absolute neutrophil count 1.5×10⁹/L
   Platelet count 100×10⁹/L
   Hemoglobin 9 g/dL
AST and ALT <3× upper limit of Normal [ULN](or <5×ULN in case of liver involvement). Total bilirubin <1.5×ULN (or <2×ULN in case of liver involvement OR Gilbert's disease).
Calculated or measured creatinine clearance (CrCl) of 30 mL/minute calculated using the formula of Cockcroft and Gault ([140−Age]×Mass [kg]/[72×Creatinine mg/dL) (×0.85 if female). Twenty-four hour urine collection is not required but is allowed.
International normalized ratio (INR) or prothrombin time (PT)<1.5×ULN except for subjects receiving anticoagulation therapy, who must be on stable dose of anticoagulant therapy for 6 weeks prior to enrollment

### Exclusion Criteria

Subjects are excluded from the study if any of the following criteria apply.

### Disease Related

Untreated or symptomatic central nervous system (CNS) metastases or leptomeningeal disease.
Subjects with asymptomatic CNS metastases are eligible if clinically stable for at least 4 weeks and do not require intervention (including use of corticosteroids).
Subjects with treated brain metastases are eligible provided the following criteria are met:
   Definitive therapy was completed at least 2 weeks prior to the first planned dose of study treatment (stereotactic radiosurgery at least 7 days prior to first planned dose of study treatment)
   At least 7 days prior to first planned dose of study treatment: any CNS disease is clinically stable, subject is off steroids for CNS disease (unless steroids are indicated for a reason unrelated to CNS disease), and subject is off or on stable doses of anti-epileptic drugs

Other solid tumor cohort excludes primary tumors of the CNS, squamous non-small cell lung cancer, gastric adenocarcinoma, and gastroesophageal junction adenocarcinoma.

### Other Medical Conditions

History of other malignancy within the past 2 years, with the following exceptions:
curatively treated non-melanoma skin malignancy
cervical cancer in situ
curatively treated uterine cancer stage I
curatively treated ductal or lobular breast carcinoma in situ and not currently receiving any systemic therapy
localized prostate cancer that has been treated surgically with curative intent and presumed cured

Impaired cardiac function or clinically significant cardiac disease including: unstable angina within 6 months prior to first dose of study treatment, acute myocardial infarction <6 months prior to first dose of study treatment, New York Heart Association (NYHA) class II-IV congestive heart failure, uncontrolled hypertension (defined as an average systolic blood pressure >160 mmHg or diastolic >100 mmHg despite optimal treatment), uncontrolled cardiac arrhythmias requiring anti-arrhythmic therapy other than beta blockers or digoxin, active coronary artery disease or corrected QT interval (QTc) ≥470.

Active infection requiring systemic treatment or any uncontrolled infection within 14 days prior to first dose of study treatment.

Known human immunodeficiency virus (HIV) infection with CD4+ T-cell (CD4+) counts <350 cells/L, hepatitis C infection (subjects with hepatitis C that achieve a sustained virologic response following antiviral therapy are allowed), or hepatitis B infection (subjects with hepatitis B surface antigen [SAg] or core antibody that achieve sustained virologic response with antiviral therapy directed at hepatitis B are allowed).

History of systemic disease or ophthalmologic disorders requiring chronic use of ophthalmic steroids.

Evidence of any ongoing ophthalmologic abnormalities or symptoms that are acute (within 4 weeks) or actively progressing.

Unwillingness to avoid use of contact lenses during study treatment and for at least 100 days after the end of treatment.

Recent (within 6 months) corneal surgery or ophthalmic laser treatment or recent (within 6 months) history of, or evidence of, corneal defects, corneal ulcerations, keratitis, or keratoconus, or other known abnormalities of the cornea that may pose an increased risk of developing a corneal ulcer.

### Prior/Concomitant Therapy

Prior treatment with any investigational selective inhibitor of the FGF-FGFR pathway (unless approved standard of care for tumor indication).

Any anticancer therapy or immunotherapy within 4 weeks prior to enrollment;
Palliative radiotherapy is allowed, provided it has been completed more than 14 days prior to the first dose of study treatment
All treatment-related toxicity needs to be resolved to grade 1 prior to the first dose of study treatment, with exception of alopecia or toxicities considered irreversible (defined as having been present and stable >21 days) which are not otherwise described in the exclusion criteria

Major surgical procedure within 28 days prior to first dose of study treatment.
Minor surgery requiring local/epidural anesthesia must be completed more than 72 hours before first dose of study treatment. In all cases, the subject must be sufficiently recovered and stable before treatment administration.

### Prior/Concurrent Clinical Study Experience

Currently receiving treatment in another investigational device or drug study, or less than 28 days since ending treatment on another investigational device or drug study(ies). Other investigational procedures while participating in this study are excluded.

### Other Exclusions

Female subjects of childbearing potential unwilling to use protocol specified method of contraception see Appendix 5 (Section 11.5) during treatment and for an additional 90 days after the last dose of bemarituzumab.

Female subjects who are breastfeeding or who plan to breastfeed while on study through 90 days after the last dose of bemarituzumab.

Female subjects planning to become pregnant while on study through 90 days after the last dose of bemarituzumab.

Female subjects of childbearing potential with a positive pregnancy test assessed at screening by a highly sensitive serum pregnancy test.

Subject has known sensitivity to any of the products to be administered during dosing.

Subject likely to not be available to complete all protocol-required study visits or procedures, and/or to comply with all required study procedures to the best of the subject and investigator's knowledge.

History or evidence of any other clinically significant disorder, condition or disease (with the exception of those outlined above) that, in the opinion of the investigator or Amgen physician, if consulted, would pose a risk to subject safety or interfere with the study evaluation, procedures or completion.

Subjects falling under the vulnerable population (prisoners, institutionalized individuals, adult subjects under legal protection measures [judicial protection or guardianship measures], or others who may be considered vulnerable) unless where noted (e.g., individuals who are illiterate or visually impaired).

### Subject Enrollment

Before subjects begin participation in any study-specific activities/procedures, the following items are required: a copy of the site's written institutional review board/independent ethics committee (IRB/IEC) approval of the protocol, informed consent form, and all other subject information and/or recruitment material, if applicable.

The subject or the subject's legally authorized representative must personally sign and date the IRB/IEC and approved informed consent before commencement of study-specific procedures.

Each subject who enters into the screening period for the study (defined as when the subject signs the informed consent) receives a unique subject identification number before any study-related activities/procedures are performed. The subject identification number will be assigned via Interactive Response Technology (TRT). This number will be used to identify the subject throughout the clinical study and must be used on all study documentation related to that subject.

The subject identification number must remain constant throughout the entire clinical study; it must not be changed after initial assignment, including if a subject is rescreened.

A subject is considered enrolled when the investigator decides that the subject has met all eligibility criteria. The investigator is to document this decision and date, in the subject's medical record and in/on the Subject Enrollment Case Report Form (CRF).

### Screen Failures

Screen failures are defined as subjects who consent to participate in the main clinical study but are not subsequently enrolled in the study (during pre-screening, subjects that are not FGFR2b overexpressing will not be counted as screen failures). A minimal set of screen failure information will be collected that includes demography, screen failure details, eligibility criteria, medical history, prior therapies, and any serious adverse events. Individuals who do not meet the criteria for participation in this study (screen failure) may be rescreened.

### Bemarituzumab

Information on bemarituzumab in the study, including dosing and dosing instructions are shown in Table 3 below.

TABLE 3

| | |
|---|---|
| Dosage | Bemarituzumab is supplied as a sterile, aqueous, |
| Formulation | colorless to slightly yellowish, pyrogen-free |
| | solution supplied in single-use glass vials. The |
| | composition of the drug product contains 20 |
| | mg/mL active ingredient (supplied as a 20 mL |
| | vial [400 mg bemarituzumab per vial]), L- |
| | histidine, sucrose, and polysorbate 20 at pH 6.0. |
| | The final drug product will be provided as a |
| | solution which should be stored refrigerated (2° C. |
| | to 8° C.), protected from light, and will be diluted |
| | for administration according to the instructions |
| | provided to the site. |
| Dosage Level(s) | Dose Level 1: 22 mg/kg IV on cycle 1 day 1, |
| | then 15 mg/kg Q2W thereafter starting on day 15. |
| | Dose Level 1A: 15 mg/kg IV Q2W plus 1 |
| | additional 7.5 mg/kg dose on cycle 1 day 8 only |
| | Each cycle is 14 days. |
| Route of | IV Infusion |
| Administration | The planned dose, quantity administered, start |
| Accountability | date/time, stop date/time, lot number of |
| | investigational product, reason total quantity |
| | changed or administration withheld, reason for |
| | administration delay, and reason for infusion |
| | interruption are to be recorded on each subject's |
| | eCRF(s). |
| Dosing | Bemarituzumab will be administered as an IV |
| Instructions | infusion, under medical supervision, over |
| | approximately 30 minutes (±10 minutes) via a |
| | peripheral vein or central venous catheter. The |
| | IV administration set for bemarituzumab infusion |
| | must contain a 0.22 μm in-line filter. |
| | Bemarituzumab dosing should occur every |
| | 14 days (±3 days) of the scheduled visit date |
| | for cycles 1 through 3. Starting at cycle 4, if |
| | needed, doses may be delayed up to 7 days. |
| | Doses should not be administered fewer than |
| | 7 days apart. |

eCRF = electronic case report form;
IV = intravenous;
Q2W = every 2 weeks

### Dose Level Determination

Two dose levels may be explored in Part 1: Dose Level 1 (22 mg/kg IV cycle 1 day 1, followed by 15 mg/kg IV Q2W thereafter starting on day 15) and Dose Level 1A (15 mg/kg IV Q2W plus 1 additional 7.5 mg/kg dose on cycle 1 day 8). Part 1 of the study will start with Dose Level 1 (22 mg/kg IV cycle 1 day 1, followed by 15 mg/kg IV Q2W thereafter starting on day 15). The study DLT evaluation period is 28 days. Once 3 to 6 subjects have completed the DLT period, a Dose Level Review Team (DLRT) meeting will be convened.

In Part 1, the DLRT will use guidelines based on an mTPI-2 design. The mTPI-2 escalation/de-escalation guideline for each dose cohort is derived with a target toxicity probability of 0.25, acceptable toxicity probability interval of (0.20, 0.30). A dose level will be considered unsafe, with no additional subjects enrolled at that dose level, if it has an estimated 95% or more probability of exceeding the target DLT (i.e., the elimination boundary). The specific guidelines are described below:
Subjects in the first treatment group are treated at Dose Level 1
To assign a dose to the next treatment group of subjects, the guidelines in Table 4 will be used.
“Eliminate” means that the current and higher doses are eliminated from the trial to prevent treating any future subjects at these doses because they are overly toxic.
When a dose is eliminated, de-escalation to Dose Level 1A occurs. When the lowest dose is eliminated, the trial is stopped for safety.
When at a lower dose and the rule indicates dose escalation, treat the new subjects at a higher dose.
If none of the actions (i.e., de-escalation, or elimination) is triggered, new subjects are treated at the current dose.
If the current dose is the lowest dose and the rule indicates dose de-escalation, new subjects are treated at the lowest dose unless the number of DLTs reaches the elimination boundary, at which point the trial is stopped for safety.
If the current dose is the highest dose and the rule indicates dose escalation, treat the new patients at the highest dose.

The above steps are repeated until the maximum sample size of 18 is reached or the number of subjects treated at the current dose reaches 9, and the decision according to Table 4 is to stay at the current dose.

TABLE 4
Dose Escalation/De-escalation Rules for the mTPI-2 Design

| | Subjects treated at the current dose | | | | | | | | |
|---|---|---|---|---|---|---|---|---|---|
| | 1 | 2 | 3 | 4 | 5 | 6 | 7 | 8 | 9 |
| Escalate if # of DLT ≤ | 0 | 0 | 0 | 0 | 0 | 1 | 1 | 1 | 1 |
| De-escalate if # of | 1 | 1 | 1 | 2 | 2 | 2 | 3 | 3 | 3 |
| DLT ≥ | | | | | | | | | |
| Eliminate if # of | NA | NA | 3 | 3 | 3 | 4 | 4 | 4 | 5 |
| DLT ≥ | | | | | | | | | |

DLT = dose limiting toxicity; mTPI = modified toxicity probability interval; NA = not applicable
# of DLT is the number of subjects with at least 1 DLT.
“NA” means that a dose cannot be eliminated before treating 3 subjects.
When none of the actions (i.e., escalate, de-escalate, or eliminate) is triggered, stay at the current dose for treating the next cohort of subjects.

### Dose Limiting Toxicities

Dose limiting toxicities are defined as any of the following adverse events during the DLT evaluation period (28 days) considered by the investigator to be at least possibly related to bemarituzumab:
Grade 4 neutropenia of any duration
Febrile neutropenia
Grade 4 thrombocytopenia
Grade 3 thrombocytopenia with grade >2 bleeding or lasting >7 days
Grade 4 anemia
Grade 5 toxicity (e.g., death not due to disease progression)
Any grade 3 ophthalmologic adverse event that does not resolve within 3 days
Any grade 4 ophthalmologic adverse event
Any grade 4 laboratory value
Any grade 4 vomiting or diarrhea
Grade 3 vomiting or Grade 3 diarrhea lasting more than 3 days despite optimal medical support
Grade >3 nausea lasting 3 days or more despite optimal medical support
Grade 3 fatigue lasting 1 week or longer
Any subject meeting the criteria for Hy's Law case (i.e., severe drug-induced liver injury [DILI]) will be considered a DLT. A Hy's Law case is defined as: AST or ALT values of ≥3×ULN AND with serum total bilirubin (TBIL) level of >2× ULN or INR >1.5 without signs of cholestasis and with no other clear alternative reason to explain the observed liver related laboratory abnormalities (see Section 11.7 for further explanation of Hy's law case and Management of Hepatic Function).
Any other grade ≥3 adverse event with the following exceptions:
   DLT Exemption: Asymptomatic grade 3 amylase and/or lipase lasting <72 hours, and without radiological signs of pancreatitis
   DLT Exemption: Asymptomatic grade 3 electrolyte abnormalities that last <72 hours, are not clinically complicated, and resolve spontaneously or respond to medical interventions
   DLT Exemption: Other select lab abnormalities that do not appear to be clinically relevant or harmful to the subject (e.g., grade 3 lymphopenia, grade 3 hypoalbuminemia), and/or can be corrected with replacement or modifications
   DLT Exemption: A transient (resolving to grade <1 within 6 hours of onset) grade 3 infusion-related adverse event

Subjects enrolled in dose exploration may be replaced if they are not evaluable for a DLT (e.g., a subject did not receive planned study treatment [100% of planned doses of bemarituzumab] or ended the study treatment before completion of DLT evaluation period for a reason other than experiencing a DLT). The replaced subject may continue on study at the Investigator's discretion and after discussion with the Medical Monitor. Dosing for an individual will be stopped for any occurrence of a DLT or if criteria are met.

### Bemarituzumab Dosage Adjustments, Delays, Rules for Withholding or Restarting, Permanent Discontinuation

Bemarituzumab doses may be held for bemarituzumab-related adverse events following the guidelines outlined in Tables 5.1, 5.2, and 5.3. The reason for dose delay of bemarituzumab is to be recorded on each subject's CRFs.

After cycle 1, the bemarituzumab dose should be recalculated only if the weight changes >10% from the cycle 1 day 1 weight. If the dose is recalculated due to a >10% weight change from cycle 1 day 1, the weight used for the recalculated dose should function as the new baseline for subsequent evaluation of dose recalculations.

If dose reductions or interruptions that do not fall within these guidelines are being considered by the investigator, these will require discussion with the Sponsor or designee.

Cycles may be delayed to manage toxicity. Any cycle delays of longer than 21 days, regardless of reason, should be discussed with the medical monitor prior to re-initiation.

### Corneal Events

Any subject with a corneal event which occurs within 100 days of last receiving a dose of bemarituzumab, regardless if deemed related or not related to bemarituzumab, should be evaluated by an ophthalmologist. Any subject who reports pain or irritation of the eye or change in vision should be evaluated by an ophthalmologist. Tables 5.1, 5.2, and 5.3 provide additional guidance.

TABLE 5.1
Bemarituzumab Dose Modification Guidelines for Adverse Events

| | | | | |
|---|---|---|---|---|
| | | | Re-start | Permanent |
| Grade | Interruption/Delay | Specific Management | Guidance | Discontinuation |

Corneal Toxicity^{a}

| | | | | |
|---|---|---|---|---|
| 1 | n/a | n/a | n/a | n/a |
| 2 | Clinical judgement | Refer to ophthalmologist | If held/delayed, | Discontinue if |
| | required. If subject | for evaluation | resume at full | grade 3 or higher |
| | has not been seen by | Treatment as deemed | dose if event | event recurs with |
| | ophthalmologist for | appropriate by the | has improved to | restart of the |
| | the Grade 2 event, | ophthalmologist. | grade ≤1. | drug. |
| | treatment should | Bemarituzumab may be | | |
| | either be held/delayed | continued or held, based | | |
| | until ophthalmologic | on ophthalmologic | | |
| | evaluation or, if not | findings. | | |
| | associated with a | | | |
| | change in vision, | | | |
| | subject may receive a | | | |
| | maximum of 1 dose | | | |
| | prior to | | | |
| | ophthalmologic | | | |
| | evaluation. | | | |
| 3 | Immediate | Refer to ophthalmologist | Resume at full | Discontinue |
| | interruption/delay | for evaluation urgently. | dose if event | treatment if event |
| | until event has | Treatment as deemed | has improved to | does not improve |
| | improved to grade ≤1 | appropriate by the | grade ≤1. | to grade ≤1 |
| | | ophthalmologist. | | Discontinue if |
| | | Close follow-up by | | grade 3 or higher |
| | | ophthalmologist until | | event recurs with |
| | | event has improved to | | restart of the |
| | | grade ≤1. | | drug. |
| 4 | n/a | Refer to ophthalmologist | n/a | Discontinue |
| | | for evaluation urgently. | | bemarituzumab |
| | | Treatment as deemed | | |
| | | appropriate by the | | |
| | | ophthalmologist. | | |
| | | Close follow-up by | | |
| | | ophthalmologist until | | |
| | | event has improved to | | |
| | | grade ≤1. | | |

TABLE 5.2
Bemarituzumab Dose Modification Guidelines for Adverse Events

| | | | | |
|---|---|---|---|---|
| CTCAE | Interruption/ | | | Permanent |
| Grade | Delay | Specific Management | Re-start guidance | Discontinuation |

Infusion-related Reaction

| | | | | |
|---|---|---|---|---|
| 1 | Decrease | Increase monitoring of | n/a | n/a |
| | rate of | vital signs as medically | | |
| | infusion by | indicated until the | | |
| | 50% | subject is deemed | | |
| | | medically stable. | | |
| | | Treat per institutional | | |
| | | guidelines. | | |
| 2 | Immediate | Increase monitoring of | After all symptoms | For subjects who |
| | interruption/ | vital signs as medically | have resolved: | develop grade 2 |
| | delay until | indicated until the | administer | infusion-related |
| | event has | subject is deemed | premedication (e.g., | reaction despite |
| | improved to | medically stable. | antihistamines, | adequate |
| | grade ≤1 | Treat per institutional | corticosteroids, | premedication, |
| | | guidelines. Additional | and acetaminophen) | permanently |
| | | appropriate medical | 1.5 hours (±30 | discontinue |
| | | therapy may include but | minutes) prior | bemarituzumab. |
| | | is not limited to IV | to infusion using | |
| | | fluids, antihistamines, | institutional | |
| | | NSAIDs, acetaminophen, | standards resume | |
| | | and narcotics. | bemarituzumab | |
| | | | at reduced | |
| | | | infusion rate | |
| | | | (50% or less of | |
| | | | standard rate) | |
| 3 or 4 | Immediate | Increase monitoring of | n/a | Permanently |
| | interruption | vital signs as medically | | discontinue |
| | | indicated until the | | bemarituzumab |
| | | subject is deemed | | |
| | | medically stable. | | |
| | | Hospitalization may be | | |
| | | indicated. | | |
| | | Treat per institutional | | |
| | | guidelines. Additional | | |
| | | appropriate medical | | |
| | | therapy may include but | | |
| | | is not limited to IV | | |
| | | fluids, antihistamines, | | |
| | | NSAIDS, acetaminophen, | | |
| | | narcotics, oxygen, | | |
| | | vasopressors, | | |
| | | corticosteroids, and | | |
| | | epinephrine. In cases of | | |
| | | anaphylaxis, epinephrine | | |
| | | should be used | | |
| | | immediately. | | |

TABLE 5.3
Bemarituzumab Dose Modification Guidelines for Adverse Events

| | | | | |
|---|---|---|---|---|
| | Interruption/ | Specific | | Permanent |
| CTCAE Grade | Delay | Management | Re-start guidance | Discontinuation |

All other bemarituzumab-related adverse events

| | | | | |
|---|---|---|---|---|
| 1 or 2 | n/a | Treat per | n/a | n/a |
| | | institutional | | |
| | | guidelines. | | |
| 3 (first occurrence) | Delay or miss | Treat per | If recovery to | n/a |
| | dose until | institutional | baseline or grade 1 | |
| | recovery to | guidelines. | within 28 days of | |
| | baseline or | | onset of event, may | |
| | grade 1 | | resume at full dose | |
| 3 (second | Delay or miss | Treat per | Resume at full dose if | n/a |
| occurrence) | dose until | institutional | event has improved to | |
| | recovery to | guidelines. | grade ≤1 within | |
| | baseline or | | 28 days of onset of | |
| | grade 1 | | event | |
| Grade 3 (third | Immediate | Treat per | n/a | Permanently |
| occurrence) | interruption | institutional | | discontinue |
| Grade 3 which does | | guidelines. | | bemarituzumab |
| not recover to | | | | |
| baseline or grade 1 | | | | |
| within 21 days of | | | | |
| onset event | | | | |
| Any grade 4 | | | | |

CTCAE = Common Terminology Criteria for Adverse Events;
IV = intravenous;
N/A = not applicable;
NSAIDs = nonsteroidal anti-inflammatory drugs;
^{a} Ocular adverse events that are not corneal toxicity should follow dose modifications for all other bemarituzumab related adverse events.

### Prior Treatment

All prior medications must be recorded in the designated eCRF forms. Prior therapies that were being taken/used 3 months prior to enrollment will be collected.

For prior anticancer therapies for the cancer being studied, therapy name, setting, dose, unit, frequency, start date, stop date, best response, and reason for discontinuation dating back to initial diagnosis are collected. For anticancer therapies including multiple individual components, information for each component should be collected. For all other prior therapies, therapy name, indication, dose, unit, frequency, route, start date, and stop date are collected.

### Concomitant Treatment

Throughout the study, investigators may prescribe any concomitant medications or treatments deemed necessary to provide adequate supportive care except for certain treatments.

To mitigate the risk of corneal toxicities, prophylactic use of ocular lubricants and eyelid hygiene is recommended. Ocular lubricants (e.g., preservative free artificial tears) should be self-administered at least 3 times daily throughout the treatment period and for 28 (+3) days after the last dose. They may be polyvinyl alcohol or liquid polyol based. If preservative free is not available, formulations with preservatives are allowed. Methylcellulose-based lubricants should not be used. Viscous lubricants which can cause blurriness should be avoided.

Concomitant therapies are to be collected from informed consent through the end of SFU, with the exception of ophthalmologic and anticancer therapies, which are collected through LTFU. For concomitant therapies, including vaccines, therapy name, indication, dose, unit, frequency, route, start date, and stop date are collected. For subsequent anticancer therapies taken for the cancer under study, drug name, start date, and stop date are collected.

### Discontinuation of Study Treatment and Subject Discontinuation/Withdrawal

Subjects have the right to withdraw from investigational product and/or other protocol required therapies, protocol procedures, or the study as a whole at any time and for any reason without prejudice to their future medical care by the physician or at the institution.

The investigator and/or sponsor can decide to withdraw a subject(s) from investigational product, device, and/or other protocol-required therapies, protocol procedures, or the study as a whole at any time prior to study completion.

### Discontinuation of Study Treatment

Subjects (or a legally authorized representative) can decline to continue receiving investigational product and/or other protocol-required therapies and/or procedures at any time during the study but continue participation in the study. If this occurs, the investigator is to discuss with the subject the appropriate processes for discontinuation from investigational product or other protocol-required therapies and must discuss with the subject the possibilities for continuation of the Schedule of Activities (see FIG. 4) including different options of follow-up (e.g., in person, by phone/mail, through family/friends, in correspondence/communication with other treating physicians, from the review of medical records) and collection of data, including endpoints, adverse events, and must document this decision in the subject's medical records. Subjects who have discontinued investigational product and/or other protocol-required therapies and/or procedures should not be automatically removed from the study. Whenever safe and feasible, it is imperative that subjects remain on-study to ensure safety surveillance and/or collection of outcome data.

Reasons for early removal from protocol-required investigational product(s) or procedural assessments may include any of the following:
decision by Sponsor
lost to follow-up
death
adverse event
subject request
ineligibility determined
protocol deviation
non-compliance
disease progression
   radiographic progression (RECIST v1.1)
   clinical progression (investigator's assessment)
requirement for alternative therapy
pregnancy

### Subject Discontinuation/Withdrawal From the Study

Withdrawal of consent for a study means that the subject does not wish to receive further protocol-required therapies or procedures, and the subject does not wish to or is unable to continue further study participation. Subject data up to withdrawal of consent will be included in the analysis of the study, and where permitted, publicly available data can be included after withdrawal of consent. The investigator is to discuss with the subject appropriate procedures for withdrawal from the study and must document the subject's decision to withdraw in the subject's medical records.

If a subject withdraws from the study, he/she may request destruction of any samples taken and not tested.

Reasons for removal of a subject from the study include the following: decision by sponsor, withdrawal of consent from study, death, and lost to follow-up.

### Lost to Follow-up

A subject will be considered lost to follow-up if he or she repeatedly fails to return for scheduled visits and is unable to be contacted by the study site.

The following actions must be taken if a subject fails to return to the clinic for a required study visit:
The site must attempt to contact the subject and reschedule the missed visit as soon as possible and counsel the subject on the importance of maintaining the assigned visit schedule and ascertain whether or not the subject wishes to and/or is able to continue in the study.
In cases in which the subject is deemed lost to follow-up, the investigator or designee must make every effort to regain contact with the subject (where possible, 3 telephone calls and, if necessary, a certified letter to the subject's last known mailing address or local equivalent methods). These contact attempts are to be documented in the subject's medical record.
If the subject continues to be unreachable, he/she will be considered to have withdrawn from the study with a primary reason of lost to follow-up.
For subjects who are lost to follow-up, the investigator should search publicly available records where permitted to ascertain survival status. This ensures that the data set(s) produced as an outcome of the study is/are as comprehensive as possible.

### Efficacy Assessments

### Radiological Imaging Assessment

The extent of disease will be evaluated by contrast-enhanced computed tomography (CT)/magnetic resonance imaging (MRI) according to RECIST v1.1. All radiological imaging will be performed as per institutional standards. In order to reduce radiation exposure for subjects, low dose CT should be utilized whenever possible.

### Screening Scans:

The screening scans should be performed within 28 days (the scans may be performed within 31 days) prior to cycle 1 day 1 and include clinical examination and appropriate imaging techniques (preferably CT scans with appropriate slice thickness per RECIST v1.1; MRIs are acceptable). If there are multiple screening scans, the one closest to the enrollment date will be used as baseline.

Radiological assessment must include CT/MRI (with contrast) of the chest, abdomen and pelvis, as well as assessment of all other known sites of disease. Tumor response assessment will be performed by the investigator per RECIST v1.1 guidelines (Section 11.9).

All subjects with brain metastasis must have MRI of the brain performed. All brain scans for subjects with brain metastasis are required to be MRI unless MRI is contraindicated, and then CT with contrast is acceptable. Brain imaging (MRI or CT) should be performed if signs or symptoms suggestive of CNS metastases are present.

### Subsequent Scans:

All subsequent scans should be performed in the same manner (e.g., with the same contrast, MRI field strength) as at screening, ideally on the same scanner. If the imaging modality must be altered (e.g., unscheduled assessment) consultation with the medical monitor is recommended.

During treatment and follow-up, radiological imaging of the chest, abdomen, pelvis, as well as all other known sites of disease, will be performed independent of treatment cycle as specified in the Schedule of Activities (see FIG. 4). Imaging may also be performed more frequently if clinically necessitated at the discretion of the managing physician. Radiologic imaging and tumor assessment will be performed until start of new anticancer therapy, disease progression, death, withdrawal of consent, or end of study, whichever occurs first.

Determination of disease response for clinical management and response evaluation will be performed at the clinical sites per RECIST v1.1. Confirmation of response by a repeat scan is required after 4 weeks from the first documentation of response. Scans for intrahepatic cholangiocarcinoma, cervical cancer, and head and neck cancer may be submitted to a central imaging core laboratory for archival, and, if needed, response assessment including RECIST v1.1, and/or exploratory analysis (e.g., volumetric and viable tumor measurements).

### Serum Tumor Markers

Serum tumor markers specific to each tumor type should be collected according to the Schedule of Activities (see FIG. 4). Tumor markers found to be elevated at baseline must normalize for confirmation of radiologic CR. CA-125 should be collected in ovarian cancer subjects within 2 weeks of screening. A CA-125 response is defined as at least a 50% reduction in CA-125 from a pretreatment sample. The CA-125 response in ovarian cancer subjects must be confirmed and maintained for at least 28 days.

### Safety Assessments

Planned time points for all safety assessments are shown in FIG. 4

### Vital Signs

The following measurements must be performed: systolic/diastolic blood pressure, heart rate, respiratory rate, and temperature. Subject must be seated in a rested and calm state for at least 5 minutes before blood pressure assessments are conducted. The position selected for a subject should be the same that is used throughout the study and documented on the vital signs CRF. The temperature location selected for a subject should be the same that is used throughout the study and documented on the vital signs CRF. All measurements are recorded on the vital signs CRF.

### Electrocardiograms (ECGs)

Subject must be in supine position in a rested and calm state for at least 5 minutes before ECG assessment is conducted. If the subject is unable to be in the supine position, the subject should be in most recumbent position as possible. The ECG must include the following measurements: Heart Rate, QRS, QT, QTc, and PR intervals. The PI or designated site physician will review all ECGs. Once signed, the original ECG tracing will be retained with the subject's source documents.

### Clinical Laboratory Assessments

The investigator is responsible for reviewing laboratory test results and recording any clinically relevant changes occurring during the study in the Events CRF. The investigator must determine whether an abnormal value in an individual study subject represents a clinically significant change from the subject's baseline values. In general, abnormal laboratory findings without clinical significance (based on the investigator's judgment) are not to be recorded as adverse events. However, laboratory value changes that require treatment or adjustment in current therapy are considered adverse events. Where applicable, clinical sequelae (not the laboratory abnormality) are to be recorded as the adverse event.

All protocol-required laboratory assessments must be conducted in accordance with the Schedule of Activities (FIG. 4).

### Vital Status

Vital status must be obtained for all subjects within the limits of local law. This includes subjects who may have discontinued study visits with or without withdrawing consent and should include interrogation of public databases, if necessary. If deceased, the date and reported cause of death should be obtained.

### Ophthalmologic Examinations

Ophthalmologic examinations will be performed according to the Schedule of Activities (FIG. 4). Ophthalmologic adverse events of any grade occurring up to 100 days after the last dose of bemarituzumab should be reported by the investigator.

The ophthalmologic examination should include distance corrected visual activity of each eye separately with acuity recorded as the log MAR equivalent, slit lamp examination of the anterior segment, tonometry (intraocular pressure measurement), and ocular surface staining (e.g., fluorescein). In addition, a dilated retinal examination or 3 field retinal photographs should be performed at screening and at every other ophthalmic evaluation. Additionally, optical coherence tomography (OCT) of the macula is required specifically for subjects with diagnosed or suspected retinal pigment epithelium (RPE) detachment. All assessments will be done according to local practice.

Distance corrected visual acuity will be assessed using Snellen or Landolt C Chart and acuity must be recorded as log MAR equivalent. The same method should be used at screening and throughout the study. Other visual acuity charts may be utilized, but must be pre agreed with the medical monitor.

Ophthalmologic examinations should be performed regardless of dose delays per the Schedule of Activities (FIG. 4). The ophthalmological examination may be repeated at any time, as clinically indicated. After the SFU visit if the subject has any persistent ophthalmologic findings, the assessments should continue until resolution of findings, withdrawal of consent, death, or loss to follow-up. Ocular adverse events should be monitored by an ophthalmologist until resolution.

The following measures will be implemented for ophthalmologic adverse events:
Manage as per standard of care
Dose modification and interruption guidelines for the investigator are provided in (Table 5.1)

If any clinically significant changes are noted compared to the previous examination or, if the subject has any grade 2 or higher ocular signs or symptoms, the subject should be evaluated by an ophthalmologist.

### Adverse Events

The adverse event grading scale to be used for this study will be the Common Terminology Criteria for Adverse Events (CTCAE) Version 5.0.

The investigator is responsible for ensuring that all adverse events observed by the investigator or reported by the subject that occur after first dose of investigational product through the end of SFU are reported using the Events CRF.

### Serious Adverse Events

The investigator is responsible for ensuring that all serious adverse events observed by the investigator or reported by the subject that occur after signing of the informed consent through 28 (+3) days after the last day of the dosing interval of investigational product are reported using the Events CRF.

All serious adverse events will be collected, recorded and reported to the sponsor or designee within 24 hours of the investigator's awareness of the event. The investigator will submit any updated serious adverse event data to the sponsor within 24 hours of it being available.

Since the criteria of the CTCAE grading scale differs from the regulatory criteria for serious adverse events, if adverse events correspond to grade 4 CTCAE toxicity grading scale criteria (e.g., laboratory abnormality reported as grade 4 without manifestation of life threatening status), it will be left to the investigator's judgment to also report these abnormalities as serious adverse events. For any adverse event that applies to this situation, comprehensive documentation of the event's severity must be recorded in the subject medical records.

If the investigator becomes aware of serious adverse events suspected to be related to IP or any fatal adverse event (regardless of causality) after the protocol required reporting period is complete, then these serious adverse events will be reported within 24 hours following the investigator's awareness of the event on the Events CRF.

There is no requirement to actively monitor study subjects after the study has ended with regards to study subjects treated by the investigator. However, if the investigator becomes aware of serious adverse events suspected to be related to investigational product after the protocol-required reporting period is complete, then these serious adverse events will be reported within 24 hours following the investigator's awareness of the event on the Events CRF.

As one of the study endpoints is overall survival, the investigator is responsible for ensuring that all fatal serious adverse events (regardless of causality) are collected and reported within 24 hours of awareness.

Serious adverse events reported outside of the protocol-required reporting period will be captured within the safety database as clinical trial cases and handled accordingly based on relationship to investigational product.

If further safety related data is needed to fulfill any regulatory reporting requirements for a reportable event, then additional information may need to be collected from the subject's records after the subject ends the study.

### Follow-Up of Adverse Events and Serious Adverse Events

After the initial adverse event/serious adverse event report, the investigator is required to proactively follow each subject at subsequent visits/contacts. All adverse events and serious adverse events will be followed until resolution, stabilization, until the event is otherwise explained, or the subject is lost to follow-up.

All new information for previously reported serious adverse events must be reported within 24 hours following awareness of the new information. If specifically requested, the investigator may need to provide additional follow up information, such as discharge summaries, medical records, or extracts from the medical records. Information provided about the serious adverse event must be consistent with that recorded on the Events CRF.

### Regulatory Reporting Requirements for Serious Adverse Events

If subject is permanently withdrawn from protocol-required therapies because of a serious adverse event, this information must be submitted to Amgen.

Prompt notification by the investigator to the sponsor of serious adverse events is essential so that legal obligations and ethical responsibilities towards the safety of subjects and the safety of a study treatment under clinical investigation are met.

The sponsor has a legal responsibility to notify both the local regulatory authority and other regulatory agencies about the safety of a study treatment under clinical investigation. The sponsor will comply with country-specific regulatory requirements relating to safety reporting to the regulatory authority, IRBs/IECs, and investigators.

Individual safety reports must be prepared for suspected unexpected serious adverse reactions according to local regulatory requirements and sponsor policy and forwarded to investigators as necessary.

An investigator who receives an individual safety report describing a serious adverse event or other specific safety information (e.g., summary or listing of serious adverse events) from the sponsor will file it along with the Investigator's Brochure and will notify the IRB/IEC, if appropriate according to local requirements.

For studies in which the treatment assignment is blinded, to comply with worldwide reporting regulations for serious adverse events, the treatment assignment of subjects who develop serious, unexpected, and related adverse events may be unblinded before submission to regulatory authorities. Aggregate analyses may also be unblinded by the Safety Assessment Team, as appropriate. Investigators will receive notification of related serious adverse events reports sent to regulatory authorities in accordance with local requirements.

### Adverse Events of Special Interest

Selected adverse events known as bemarituzumab Events of Special Interest are ocular adverse events of any grade or seriousness occurring up to 100 days after the last dose of bemarituzumab and should be collected as adverse events. Ocular adverse events (including corneal adverse events) should be graded using a Ocular Toxicity Grading scale. Subjects should be assessed for possible bemarituzumab Events of Special Interest prior to each dose.

### Pregnancy and Lactation

Details of all pregnancies and/or lactation in female subjects will be collected after the start of study treatment and until 90 days after the last dose of bemarituzumab.

Abnormal pregnancy outcomes (e.g., spontaneous abortion, fetal death, stillbirth, congenital anomalies, and ectopic pregnancy) are considered serious adverse events.

A highly sensitive (serum) pregnancy test should be completed at screening and within 72 hours of initiation of investigational product for females of childbearing potential.

Females who have undergone a bilateral tubal ligation/occlusion should have pregnancy testing per protocol requirements. (If a female subject, or the partner of a male subject, becomes pregnant it must be reported on a Pregnancy Notification Form).

Additional pregnancy testing should be performed on day 1 of every other cycle during treatment and after discontinuing protocol required therapies at day 28 (+3) (SFU), day 56 (±3), and day 75 (+3) from the last bemarituzumab dose.

Additional on-treatment pregnancy testing may be performed at the investigator's discretion or as required per local laws and regulations.

### Pharmacokinetic Assessments

Whole blood samples will be collected for measurement of serum concentrations of bemarituzumab as specified in the Schedule of Activities (FIG. 4). Instructions for the collection and handling of biological samples will be provided by the sponsor. The actual date and time (24-hour clock time) of each sample will be recorded.

### Pharmacogenetic Assessments

If the subject consents to the optional pharmacogenetic portion of this study, DNA analyses may be performed. These optional pharmacogenetic analyses focus on inherited genetic variations to evaluate their possible correlation to the disease and/or responsiveness to the therapies used in this study. This optional assessment is separate from genomic analysis of somatic mutations in the tumor and circulating tumor DNA (ctDNA) samples included as part of the main study. The goals of the optional studies include the use of genetic markers to help in the investigation of cancer and/or to identify subjects who may have positive or negative response to investigational product. No additional samples are collected for this part of the study. For subjects who consent to this/these analysis/analyses, DNA may be extracted.

### Antibody Testing Procedures

Blood sample(s) for antibody testing are to be collected according to the time points specified in the Schedule of Activities (FIG. 4) for the measurement of anti-bemarituzumab antibodies. Samples testing positive for binding antibodies may be further characterized.

Subjects who test positive for antibodies at the final scheduled antibody time point and have clinical sequelae that are considered potentially related to an anti-bemarituzumab antibody response will also be asked to return for additional follow-up testing. This testing is to occur approximately every 3 months from the final scheduled antibody time point and continue until: (1) antibodies are no longer detectable; or (2) the subject has been followed for a period of at least 1 year (±4 weeks) post administration of bemarituzumab. More frequent testing or testing for a longer period of time may be requested in the event of safety-related concerns.

### Biomarkers

Biomarkers are objectively measured and evaluated indicators of normal biologic processes, pathogenic processes, or pharmacologic responses to a therapeutic intervention.

### Biomarker Assessment to Determine Eligibility

Provision of archival tumor biopsy/resection (bone biopsies and cytology samples are excluded) formalin fixed and embedded in paraffin (within 5 years or fresh biopsy if archival sample is not available) for prescreening FGFR2b overexpression status by IHC is required and subjects must consent to tumor tissue analysis. If FGFR2b overexpression is confirmed, PD-L1 will also be tested on remaining pre-screening tissue, if available, in head and neck squamous cell carcinoma, triple negative breast cancer, lung adenocarcinoma, cervical cancer, and endometrial cancer.

Subjects will be selected for enrollment based on FGFR2b overexpression status, as determined by a clinical trial assay (CTA) IHC assay at a central Clinical Laboratory Improvement Amendments (CLIA)-certified laboratory (Roche Tissue Diagnostics, Tucson, Arizona, US) that meets United States (US) regulatory requirements. Timing of sample collection is described in the Schedule of Activities (FIG. 4). Primary tumor and metastatic sites of specimen collection are allowed. If fresh tissue samples need to be collected, these samples should be obtained following local standard of care procedures that are not expected to present any additional significant risk to the health, safety, and welfare of the subject.

The CTA used to select subjects is the VENTANA FGFR2b (FPR2 D) assay, an IHC test to determine the FGFR2b overexpression status (positive, negative) in neoplastic tissue. Samples that are deemed positive for FGFR2b overexpression status exhibit moderate (2+) to strong (3+) membrane staining in tumor cells.

Subjects who are deemed negative for FGFR2b overexpression status (absence of moderate or strong membrane staining in tumor cells) using IHC will not be eligible for enrollment. It is the responsibility of each investigator to obtain an adequate tumor specimen for analysis of FGFR2b status for enrollment. Blocks are preferred, if available, but in lieu of blocks, unstained slides are recommended. The tumor specimen submitted should be of sufficient quantity to allow for IHC analysis; see the Laboratory Manual for details. Tumor slide or tumor block specimen processing, labeling, and shipping instructions are detailed in the Laboratory Manual that will be distributed with the specimen collection kit.

Once tumor specimens are received, analysis will be performed as efficiently as possible, and FGFR2b overexpression status results will be communicated back to the investigator or designee. Programmed death ligand-1 testing will be performed on leftover pre-screening tissue, if available, in the indications listed in FIG. 4. This PD-L1 testing is a pre-screening assessment performed in order to understand PD-L1 expression and overlap with FGFR2b, but the results of the testing will not determine eligibility.

### Biomarker Discovery

Samples will be collected to develop or address biomarker hypotheses related to bemarituzumab activity (e.g., to evaluate potential biomarkers that may correlate with treatment response). These samples may also be used for developing methods that enable better understanding of the disease.

Blood and tissue will be collected for biomarker discovery at the time points specified in the Schedule of Activities (FIG. 4), if allowed according to local regulations and agreed by Ethics Committees (EC)/Institutional Review Board (IRB). Blood samples will be collected and assessed for circulating tumor/cell-free DNA mutational profiles for potential association with clinical endpoints. Circulating tumor DNA plasma analysis will include the mandatory paired analysis of subject blood samples to identify and select out germline variants, to help refine and determine tumor-specific mutations. The circulating tumor/cell-free DNA (ctDNA) assessments are used for profiling of somatic mutations. Germline mutational results will not be reported.

When tumor samples or optional biopsy samples are available, after additional consent is provided, they may be used to examine protein expression, RNA and DNA gene expression, or somatic (tumor) mutation analysis. When tumor tissues are available, analyses of tumor specific mutations or epigenetic changes may be performed (e.g., somatic mutations). Exploratory genomic analyses of tumor tissue, or biopsies, can include the paired sequencing analysis of subject blood cell pellet samples to identify and select out germline variants, to help refine and determine tumor-specific mutations. Germline mutational results will not be reported. None of these samples will be used for screening of hereditary traits. Plasma samples outlined in FIG. 4 will be collected and biomarker analyses, such as proteomic analysis, may be performed.

### Biomarker Development/Future Research

Biomarker Development refers to using samples collected for Biomarker Discovery for future research after the study ends.

In oncology, there is particular interest in the molecular changes underlying the oncogenic processes that may identify cancer subtypes, stage of disease, assess the amount of tumor growth, or predict disease progression, metastasis, and responses to investigational product(s) or protocol-required therapies.

If consent is provided by subjects, any remaining samples collected at the time points specified in the Schedule of Activities (FIG. 4), including samples collected for biomarker assessments, may be used for future research. No additional samples will be collected for biomarker development/future research.

Test(s) may be designed to identify subjects most likely to respond positively or negatively to investigational product(s) to investigate and further understand cancer.

### Statistical Considerations

### Statistical Hypotheses

No statistical hypothesis will be tested.

### Sample Size Determination

In Part 1, there is a 92% probability of observing at least 1 DLT if the true DLT rate is 25% with 9 subjects treated at a dose level. In Part 2, the Clopper-Pearson Exact 95% lower confidence limits corresponding to observed ORRs of 11.1%, 16.7%, 22.2%, 27.8%, and 33.3% with 36 subjects are 3.1%, 6.4%, 10.1%, 14.2%, and 18.6%, respectively; subjects in Part 1 assigned to the same dose level used in Part 2 will contribute to the 36 subject total (see Table 6).

TABLE 6
95% CIs using the Clopper-Pearson Exact Method (N = 36)

| | Expected OR, n (%) | | | | |
|---|---|---|---|---|---|
| | 4 (11.1) | 6 (16.7) | 8 (22.2) | 10 (27.8) | 12 (33.3) |
| LCL (%) | 3.11 | 6.37 | 10.12 | 14.20 | 18.56 |
| UCL (%) | 26.06 | 32.81 | 39.15 | 45.19 | 50.97 |

CI = confidence interval;
LCL = lower confidence limit;
OR = objective response;
UCL = upper confidence limit.
Note:
“N” is the total sample size for a given tumor type cohort. “n” is the number of expected observed responses. The two-sided 95% confidence interval was calculated using the Clopper--Pearson Exact Method.

### Populations for Analysis

The following populations are defined:

| Population | Description |
|---|---|
| Enrolled | All enrolled subjects |
| Full Analysis Set | All subjects who received at least 1 dose |
| | of bemarituzumab |
| DLT | All subjects that are enrolled who received |
| | at least 1 dose of bemarituzumab and met either |
| | of the following: 1) experienced a DLT, or 2) |
| | on-study 28 days following first dose of |
| | investigational product |

### Covariates

The relationship between covariates and endpoints may be explored and specified in the statistical analysis plan (SAP) if appropriate.

### Subgroups

Subgroups may be explored and specified in the statistical analysis plan if appropriate.

### Statistical Analyses

The statistical analysis plan will be developed and finalized before database lock. Below is a summary of the timing and methods for the planned statistical analyses. To preserve study integrity, the final analysis will be conducted and reported following the end of study.

### Planned Analyses

### Part 1

In part 1, the DLRT will convene to review all available safety, tolerability, laboratory, and PK data during Part 1 and after Part 1 is completed (28 days following last subject enrolled in Part 1). For dose exploration decisions, the DLRT will use guidelines based on an mTPI-2 design as described in Section 6.2.1.

### Part 2

A DRT will review safety data after a specified number of subjects in the full analysis set, regardless of tumor type, have had the opportunity to be followed for 8 weeks. The specified number of subjects that will trigger a DRT review is specified in the first column of Table 7. To make their assessment, the DRT will use their clinical judgement when reviewing all relevant safety data and use the stopping guidelines in Table 7 which are based on having a >85% Bayesian posterior probability that the posterior probability of the grade 4+ treatment-related adverse event rate exceeds 20% using a beta (1,1) prior distribution.

TABLE 7
Safety Review Frequency and Stopping Boundaries
for Grade 4+ Treatment-related Adverse Events

| | Number of subjects with grade 4+ |
|---|---|
| Number of subjects that | treatment-related adverse events |
| triggers a DRT review | that triggers stopping |
| 10 | ≥4 |
| 20 | ≥6 |
| 30 | ≥9 |
| 50 | ≥13 |
| 75 | ≥19 |
| 100 | ≥24 |
| 150 | ≥35 |
| 200 | ≥46 |
| 300 | ≥67 |

### Futility

The DRT will oversee non-binding interim analyses for futility that are planned to occur after the first 12 and 24 subjects in the safety analysis set for a given tumor type cohort have had the opportunity to complete the 16-week disease assessment (two scans). Enrollment will not be paused in order to conduct the futility analyses. Stopping for futility will be based on having a <20% predictive probably that the ORR will be >15% after all 36 subjects are enrolled and have the opportunity to complete the 16-week disease assessment. A noninformative beta (1, 1) prior distribution will be used. A cohort may stop for futility if 0 out of 12 subjects or 1 out of 24 subjects have an OR (CR or PR). Table 8 provides the stopping guidelines sample sizes ranging from 11 to 35 to allow the DRT to assess multiple cohorts with different sample sizes at the same review. Operating characteristics of the stopping guidelines are described in Table 9.

TABLE 8
Stopping Guidelines

| Number of subjects evaluable | Number of ORs to conclude futility |
|---|---|
| 11 to 22 | 0 |
| 23 to 34 | 1 or less |
| 35 | 2 or less |

OR = overall response

Table Error!No text of specified style in document. Stopping Guidelines Operating Characteristics

| Assumed ORR | Probability of stopping for futility |
|---|---|
| 5% | 74.7% |
| 10% | 42.2% |
| 15% | 21.4% |
| 30% | 2.1% |
| 40% | 0.4% |

ORR = objective response rate

Before the primary analysis for the entire study, additional interim analyses will be performed by tumor cohort on select efficacy and safety endpoints once all subjects enrolled in that tumor cohort have had the opportunity to complete the 16-week disease assessment.

### Primary and Final Analyses

The primary analysis will occur when all subjects across all tumor types complete the safety follow-up visit. All efficacy and safety endpoints will be analyzed at the primary analysis. The final analysis will occur when all subjects across all tumor types complete the study. The time-to-event endpoints will be updated with further follow-up at the final analysis.

### Methods of Analyses

Part 1 and Part 2 will be analyzed separately. For Part 1, safety analyses will combine tumor types; efficacy analyses will be presented by tumor type and only if there is adequate sample size. For Part 2, efficacy analyses will be presented by tumor type; safety analyses will be presented by tumor type and overall. The primary analysis of efficacy and safety will be based on all enrolled subjects who received at least one dose of investigational product. Continuous variables will be described with the mean, median, quartiles, minimum, and maximum. Categorical data will be summarized with frequency counts and percentages. Confidence intervals (CI) for proportions will be estimated using an exact method proposed by Clopper-Pearson (Clopper and Pearson, 1934). Kaplan-Meier (KM) methods will be used to estimate the median and percentiles for time to event endpoints with CI calculated by using the Brookmeyer and Crowley method (Brookmeyer and Crowley, 1982). Kaplan-Meier methods will be used to estimate landmarks for time to event endpoints (e.g., 1-year OS) with the Greenwood formula (Kalbfleisch and Prentice, 1980) used to estimate the standard error used in CI calculation.

### Efficacy Analyses

| Endpoint | Statistical Analysis Methods |
|---|---|
| Primary | The proportion of subjects with an OR will |
| | be estimated along with a 95% CI. |
| Secondary | The proportion of subjects with disease control |
| | will be estimated along with a 95% CI. |
| | KM curves, KM quartiles with 95% CIs, and KM |
| | estimates with 95% CIs at landmark time points |
| | will be estimated for the following endpoints: |
| | duration of objective response (for only |
| | responding subjects), PFS, and OS. |
| Exploratory | Will be described in the statistical analysis |
| | plan finalized before database lock |

### Safety Analyses

| Endpoint | Statistical Analysis Methods |
|---|---|
| Primary | The incidence of DLTs, treatment-emergent adverse events, |
| | clinical laboratory abnormalities, vital signs, and |
| | corneal findings will be tabulated by dose level. |

### Adverse Events

Subject incidence of all treatment-emergent adverse events (e.g., ocular events) will be tabulated by system organ class and preferred term. Tables of grade 3 events, grade 4 events, fatal adverse events, serious adverse events, adverse events leading to interruption/withdrawal from investigational product, and treatment-emergent adverse events of interest will also be provided.

### Laboratory Test Results

The analyses of safety laboratory endpoints will include summary statistics over time. Shifts in grades of safety laboratory values the baseline and the worst on-study value will be tabulated.

### Vital Signs

The analyses of vital signs will include summary statistics over time. Shifts in vital sign values between baseline and the worst on-study value will be tabulated.

### Physical Measurements

The analyses of physical measurements will include summary statistics at baseline and possibly at select post-baseline time points.

### Electrocardiogram

The ECG measurements from this clinical study are performed as per standard of care for routine safety monitoring, rather than for purposes of assessment of potential QTc effect. Since these evaluations may not necessarily be performed under the rigorous conditions expected to lead to meaningful evaluation of QTc data; summaries and statistical analyses of ECG measurements are not planned, and these data would not be expected to be useful for meta-analysis with data from other trials.

### Antibody Formation

The incidence and percentage of subjects who develop anti-bemarituzumab antibodies at any time will be tabulated.

### Exposure to Investigational Product

The number of days on investigational product, the total dose of investigational product, and dose intensity will be summarized using descriptive statistics.

### Exposure to Concomitant Medication

Number and proportion of subjects receiving therapies of interest will be summarized by preferred term as coded by the World Health Organization Drug dictionary.

### Other Analyses

Pharmacokinetic parameters for bemarituzumab including, but not limited to, AUC, Cmax, Ctrough will be determined. Pharmacokinetics data collected from this study in combination with PK data collected from other bemarituzumab studies will be used for population PK analysis. Additional analyses will be performed to evaluate relationships between bemarituzumab exposure and selected safety or efficacy or any relevant biomarker endpoints if data are available. Details and results of these exploratory analyses will be described in separate reports.

### Example 3: Treatment of Solid Tumors with Bemarituzumab—Dose Level 1

Separate cohorts of patients having pathologically confirmed or solid tumors with FGFR2b overexpression are treated with bemarituzumab. The cohorts are organized according to tumor type as follows:
1. head and neck squamous cell carcinoma
2. triple-negative breast cancer (ER−, PR−, HER2/neu−)
3. intrahepatic cholangiocarcinoma
4. lung adenocarcinoma
5. ovarian epithelial carcinoma, including fallopian tube cancers and primary peritoneal cancers
6. endometrial adenocarcinoma
7. cervical carcinoma
8. other solid tumors

Each cohort is treated with an every two weeks (Q2W) regimen of a first administration of the bemarituzumab at a dose of 22 mg/kg, followed two weeks after the first administration and Q2W thereafter by subsequent administrations of bemarituzumab each at a dose of 15 mg/kg.

The bemarituzumab monotherapy is efficacious in treating the solid tumors in these patients as measured by objective response (defined as complete response (CR)+partial response (PR)), as determined per Response Evaluation Criteria in Solid Tumors [RECIST v1.1]).

### Example 4: Treatment of Solid Tumors with Bemarituzumab—Dose Level 2

Separate cohorts of patients having pathologically confirmed or solid tumors with FGFR2b overexpression are treated with bemarituzumab. The cohorts are organized according to tumor type as follows:
1. head and neck squamous cell carcinoma
2. triple-negative breast cancer (ER−, PR−, HER2/neu−)
3. intrahepatic cholangiocarcinoma
4. lung adenocarcinoma
5. ovarian epithelial carcinoma, including fallopian tube cancers and primary peritoneal cancers
6. endometrial adenocarcinoma
7. cervical carcinoma
8. other solid tumors

Each cohort is treated with an every two weeks (Q2W) regimen of the anti-FGFR2b antibody at a dose of 15 mg/kg, and one week after the first administration of the anti-FGFR2b antibody, administering a single subsequent administration of the anti-FGFR2b antibody at a dose of 7.5 mg/kg.

The bemarituzumab monotherapy is efficacious in treating the solid tumors in these patients as measured by objective response (defined as complete response (CR)+partial response (PR)), as determined per Response Evaluation Criteria in Solid Tumors [RECIST v1.1]).

### Example 5: Treatment of Solid Tumors with Bemarituzumab—Dose Level 1

Separate cohorts of patients having pathologically confirmed or solid tumors with FGFR2b overexpression are treated with bemarituzumab. The cohorts are organized according to tumor type as follows:
1. head and neck squamous cell carcinoma
2. triple-negative breast cancer (ER−, PR−, HER2/neu−)
3. intrahepatic cholangiocarcinoma
4. lung adenocarcinoma
5. ovarian epithelial carcinoma, including fallopian tube cancers and primary peritoneal cancers
6. endometrial adenocarcinoma
7. cervical carcinoma
8. other solid tumors

Each cohort is treated with an every two weeks (Q2W) regimen of a first administration of the bemarituzumab at a dose of 22 mg/kg, followed two weeks after the first administration and Q2W thereafter by subsequent administrations of bemarituzumab each at a dose of 15 mg/kg.

The bemarituzumab monotherapy is efficacious in treating the solid tumors in these patients as measured by progression free survival and/or overall survival.

### Example 6: Treatment of Solid Tumors with Bemarituzumab—Dose Level 2

Separate cohorts of patients having pathologically confirmed or solid tumors with FGFR2b overexpression are treated with bemarituzumab. The cohorts are organized according to tumor type as follows:
1. head and neck squamous cell carcinoma
2. triple-negative breast cancer (ER−, PR−, HER2/neu−)
3. intrahepatic cholangiocarcinoma
4. lung adenocarcinoma
5. ovarian epithelial carcinoma, including fallopian tube cancers and primary peritoneal cancers
6. endometrial adenocarcinoma
7. cervical carcinoma
8. other solid tumors

Each cohort is treated with an every two weeks (Q2W) regimen of the anti-FGFR2b antibody at a dose of 15 mg/kg, and one week after the first administration of the anti-FGFR2b antibody, administering a single subsequent administration of the anti-FGFR2b antibody at a dose of 7.5 mg/kg.

The bemarituzumab monotherapy is efficacious in treating the solid tumors in these patients as measured by progression free survival and/or overall survival.

### Example 7

A FGFR2b overexpression prevalence study was conducted procured tissues across 10 tumor indications, chosen based on the TCGA data and literature, in order to update the tumor cohorts in this study (Table 10). Based on these data tumor indications with an expected prescreening prevalence of 10% or greater (including those with 95% confidence interval including 10%) are being recruited as defined tumor cohorts. Of note, iCCA demonstrated a 1% prevalence (95% CI 0%-3%), but the majority of specimens were obtained from sites in a single country due to constraints related to rarity of the tumor type. Specimens from the other tumor types were sourced across multiple countries to ensure geographic diversity. Thus, the iCCA prevalence data from this study may not be representative of wider FGFR2b prevalence, as is supported by published literature and early results from this study. Published literature suggests FGFR2b expression (HIC staining ≥1) in ˜31% (19/62 cases) of iCCA (Junior et al, 2022). At the outset of this study, four iCCA specimens were received for pre-screening and 2/4 (50%) tested positive for any 2+3+FGFR2b levels.

TABLE 10
FGFR2b overexpression prevalence across solid
tumors (excluding gastric and squamous NSCLC)

| | Number of | Number | Prevalence of FGFR2b | |
|---|---|---|---|---|
| | specimens | positive | overexpression (95% | |
| Indication | evaluated | for FGFR2b | confidence interval) | |
| Head and neck squamous | 123 | 27 | 22% | (15%-29%) |
| cell carcinoma^{a} | | | | |
| Triple-negative | 135 | 11 | 8% | (4%-13%) |
| breast cancer^{a} | | | | |
| Intrahepatic | 188 | 2 | 1% | (0%-3%)^{a,b} |
| cholangiocarcinoma | 4 | 2 | 50% | (10%-99%)^{c} |
| Lung adenocarcinoma^{a} | 100 | 6 | 6% | (1%-11%) |
| Ovarian epithelial | 245 | 47 | 19% | (14%-24%) |
| carcinoma^{a} | | | | |
| Endometrial | 199 | 46 | 23% | (17%-29%) |
| carcinoma^{a} | | | | |
| Cervical carcinoma^{a} | 99 | 10 | 10% | (4%-16%) |
| Esophageal squamous | 197 | 8 | 4% | (1%-7%) |
| cell carcinoma^{a} | | | | |
| Pancreatic ductal | 242 | 0 | 0% | |
| adenocarcinoma^{a} | | | | |
| Colorectal | 243 | 1 | 0.4% | (0%-1%) |
| adenocarcinoma^{a} | | | | |

^{a}Procured stage III/IV FFPE tissue blocks, surgically excised or biopsied, were subjected to FGFR2b IHC using the VENTANA FGFR2b (FPR2-D) Robust Prototype Assay and any 2+/3+ staining was considered as FGFR2b overexpression.
^{b}Limited geographic diversity in these procured iCCA tissues. Majority of specimens obtained from sites in a single country.
^{c}Data for iCCA subjects. Very small sample size.

Besides gastric cancer and squamous non-small cell lung cancer (which are being studied in other bemarituzumab clinical trials), data on FGFR2b expression by immunohistochemistry (IHC) suggests overexpression (defined as subjects with FGFR2b 2+/3+ membrane staining in tumor cells) in ovarian carcinoma (19%, 95% CI 14%-24%), endometrial carcinoma (23%, 95% CI 17%-29%), squamous cell carcinoma of the head and neck (22%, 95% CI 15%-29%), cervical carcinoma (10%, 95% CI 4%-16%), triple negative breast cancer (8%, 95% CI 4%-13%), adenocarcinoma of the lung (6%, 95% CI 1%-11%), and intrahepatic cholangiocarcinoma (iCCA, 1%, 95% CI 0%-3%) (Table 10).

### Example 8: Results from a Phase 1b Basket Study Evaluating the Safety, Tolerability, Pharmacokinetics, and Efficacy of Bemarituzumab Monotherapy in Solid Tumors with FGFR2B Overexpression

Tumor samples from patients were determined to overexpress FGFR2b by immunohistochemistry (IHC) if they exhibited any moderate (2+) to strong (3+) membrane staining, according to the protocol described in Example 2. Patients with solid tumor types overexpressing FGFR2b were administered the anti-FGFR2b antibody bemarituzumab intravenously according to the protocol described in Example 2 (Part 1, phase 1b). Patients were dosed at 22 mg/kg bemarituzumab Cycle 1, day 1 (“C1/D1”), followed by 15 mg/kg bemarituzumab Q2W. The tumor types of these patients included pancreatic ductal, colorectal, ovarian, & head and neck (N=1) (pharmacokinetic data available for N=5/6 patients). Bemarituzumab serum concentration was measured at pre-specified timepoints on days 0, 1 (intensive PK sampling), 2, 4, 8, 15, 21, and 43. The preliminary PK summary for Bemarituzumab is shown in Table 11. Geometric mean Bemarituzumab Cₘₐₓ was 577 g/mL, and C_{trough} concentration was 96.5 μg/mL after cycle 1.

TABLE 11
Observed Bemarituzumab Exposures (Geometric mean: Cₘₐₓ and C_{trough}) Based on
Available Preliminary PK Data From Basket Study 20210104 (data cutoff Dec. 12, 2022)

| | C_{trough} | | | Cₘₐₓ | | | AUC336 | | | | | | | | | |
|---|---|---|---|---|---|---|---|---|---|---|---|---|---|---|---|---|
| | (μg/mL) | | | (μg/mL) | | | (hr * μg/mL) | | | t1/2 (day) | | | Tₘₐₓ (hr) | | | |
| Cycle | N | GM | CV % | N | GM | CV % | N | GM | CV % | N | GM | CV % | N | Min | Max | Median |
| 1 | 2 | 96.5 | 30.9 | 5 | 577 | 31.1 | 2 | 2540 | 25.8 | 2 | 9.7 | 43.4 | 5 | 0.75 | 6.5 | 0.75 |
| 2 | 2 | 134 | 52.8 | 2 | 377 | 30.9 | 2 | 3210 | 17.9 | 2 | 11.6 | 5.5 | 2 | 0.75 | 0.75 | 10.75 |
| 3 | | | | 2 | 407 | 58.8 | | | | | | | 2 | 0.75 | 0.75 | 0.75 |

These data indicate that for the dosing and scheduling, bemarituzumab pharmacokinetics were consistent with clinical modeling from gastric cancer indication, and that systemic bemarituzumab concentration exceeded the minimum predicted efficacious exposure threshold derived from nonclinical data (60 μg/mL).

There were no new safety findings in the patients based on known bemarituzumab safety profile.

### REFERENCES

Each of the following references is incorporated by reference in its entirety herein. 2020-2021 Basic and Clinical Science Course™ (BCSC); Section 3—Clinical Optics. Ophthalmology. https://www.aao.org/bcscsnippetdetail.aspx?id=3550b1ca-1740-4e7f-8712-70905c99eb26. 30 Jun. 2020.
Ahn S, Lee J, Hong M, et al. FGFR2 in gastric cancer: protein overexpression predicts gene amplification and high H-index predicts poor survival. Mod Pathol. 2016; 29(9):1095-1103.
Andre F, Ranson M, Dean E, et al. Abstract LB-145: Results of a Phase I Study of AZD4547, an Inhibitor of Fibroblast Growth Factor Receptor (FGFR), in Patients with Advanced Solid Tumors. AACR 104th Annual Meeting 2013, Washington, DC, American Association of Cancer Research.
BALVERSA® US Prescribing Information, 2020. https://www.janssenlabels.com/package-insert/product-monograph/prescribing-information/BALVERSA-pi.pdf
Bemarituzumab Investigator's Brochure. Thousand Oaks, CA. Amgen Inc.
Brands R C, Knierim L M, De Donno F, et al. Targeting VEGFR and FGFR in head and neck squamous cell carcinoma in vitro. Oncology Reports. 2017; 38:1877-1885.
Brookmeyer R and Crowley J. A Confidence Interval for the Median Survival Time. Biometrics. 1982; 2984; 38:29-41.
Brown A P, Courtney C L, King L M, et al. Cartilage Dysplasia and Tissue Mineralization in the Rat Following Administration of a FGF Receptor Tyrosine Kinase Inhibitor. Toxicologic Pathology. 2005; 33:449-455.
Catenacci, D V T; Rasco D; Lee J, et al. Phase I Escalation and Expansion Study of Bemarituzumab (FPA144) in Patients with Advanced Solid Tumors and FGFR2b Selected Gastroesophageal Adenocarcinoma. J Clin Oncol. 2020; 82(21):2418-2426.
Catenacci D V T, Kang Y-K, Saeed A, et al. FIGHT: A randomized, double-blind, placebo-controlled, phase II study of bemarituzumab (bema) combined with modified FOLFOX6 in 1L FGFR2b+ advanced gastric/gastroesophageal junction adenocarcinoma (GC) [Abstract]. 2021. J Clin Onc. 2021; 39(no 15_supp):4010.
Churi C R, Shroff R, Wang Y. Mutation Profiling in Cholangiocarcinoma: Prognostic and Therapeutic Implications. Plos One. 2014; 9(12):e115383.
The Common Terminology Criteria for Adverse Events, version 5.0: http://ctep. cancer.gov/protocolDevelopment/electronic_applications/ctc.htm
Clopper C J and Pearson E S. The Use of Confidence or Fiducial Limits Illustrated in the Case of the Binomial. Biometrika. 1934; 26:404-413.
Dienstmann R, Bahleda R, Barbara A, et al. First in human study of JNJ-42756493, a potent pan fibroblast growth factor receptor (FGFR) inhibitor in patients with advanced solid tumors. AACR 105th Annual Meeting 2014, San Diego, CA.
Dutt A, Salvesen H B, Chen T-H, et al. Drug-sensitive FGFR2 mutations in endometrial carcinoma. Proc Natl Acad Sci USA. 2008; 105(25):8713-8717.
Eisenhauer E A, Therasse P, Bogaerts J, et al. New response evaluation criteria in solid tumours: revised RECIST guideline (version 1.1). Eur J Cancer. 2009; 45(2):228-247.
Gatius S, Velasco A, Azueta A, et al. FGFR2 alterations in endometrial carcinoma. Modern Pathology. 2011; 24:1500-1510.
Gemo A, Deshpande A, Palencia S, et al. FPA144: A Therapeutic Antibody for Treating Patients with Gastric Cancers Bearing FGFR2 Gene Amplification. AACR 105th Annual Meeting 2014, San Diego California.
Han X, Xiao Z, and Quarles L D. Membrane and integrative nuclear fibroblastic growth factor receptor (FGFR) regulation of FGF-23. J Biol Chem. 2021; 290(16):10447-10459.
International Committee of Medical Journal Editors, Uniform Requirements for Manuscripts Submitted to Biomedical Journals: Writing and Editing for Biomedical Publication. 2013. http://www.icmje.org/
Kang X, Lin Z, Xu M, et al. Deciphering role of FGFR signalling pathway in pancreatic cancer. Cell Proliferation. 2019; 52:e12605.
Kato H, Arao T, Matsumoto K, et al. Gene amplification of EGFR, HER2, FGFR2 and MET in esophageal squamous cell carcinoma. In J Oncol. 2013; 42:1152-1158.
Katoh and Katoh. FGF signaling network in the gastrointestinal tract (review). Int J Oncol. 2006; 29(1):163-168.
Kalbfleisch J D and Prentice, R L. The Statistical Analysis of Failure Time Data, New York: John Wiley & Sons (1980)
Li P, Huang T, Zou Q, et al. FGFR2 Promotes Expression of PD-L1 in Colorectal cancer via the JAK/STAT3 Signaling Pathway. J Immunol. 2019; 202:3065-3075.
Oken M M, Creech R H, Tormey D C, et al. Toxicity and response criteria of the Eastern Cooperative Oncology Group. Am J Clin Oncol. 1982; 5(6):649-655.
PEMAZYRE® US Prescribing Information, 2021. https://www.pemazyre.com/pdf/prescribing-information.pdf
Reintjes N, Li Y, Becker A, et al. Activating Somatic FGFR2 Mutations in Breast Cancer. Plos One. 2013; 8(3):e60264.
Sequist L V, Cassier P, Varga A, et al. Phase I Study of BGJ398, a Selective Pan-FGFR Inhibitor in Genetically Preselected Advanced Solid Tumors. AACR 2014 Annual Meeting, San Diego, CA.
Shinkawa T, Nakamura K, Yamane N, et al. The Absence of Fucose but Not the Presence of Galactose or Bisecting N-Acetylglucosamine of Human IgG1 Complex-type Oligosaccharides Shows the Critical Role of Enhancing Antibody-dependent Cellular Cytotoxicity. J Biol Chem. 2003; 278(5):3466-3473.
Sun Y, Cheng Y, Zhang Y, et al. MicroRNA-889-3p targets FGFR2 to inhibit cervical cancer cell viability and invasion. Experimental and Therapeutic Medicine. 2019; 18: 1440-1448.
Tokunaga R, Imamura Y, Nakamura K, et al. Fibroblast growth factor receptor 2 expression, but not its genetic amplification, is associated with tumor growth and worse survival in esophagogastric junction adenocarcinoma. Oncotarget. 2016; 7(15):19748-19761.
Tyulyandina A, Demidova I, Gikalo M, et al. Role of FGFR2 amplification in prognosis of patients with ovarian cancer. 2018; 29(8):viii354-viii355.
Wu Y-M, Su F, Kalyana-Sundaram, et al. Identification of Targetable FGFR Gene Fusions in Diverse Cancers. Cancer Discovery. 2013; 3(6):636-647.
Xiang H, Chan A H, Ahene A, et al. Preclinical characterization of bemarituzumab, an FGFR2b antibody for the treatment of cancer. MAbs. 2021; 13(1):1981202.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a solid tumor in a subject, comprising administering to the subject an anti-FGFR2b antibody monotherapy comprising either:
(a) an every two weeks (Q2W) regimen of a first administration of the anti-FGFR2b antibody at a dose of greater than 20 mg/kg to no more than 30 mg/kg, followed two weeks after the first administration and Q2W thereafter by subsequent administrations of the anti-FGFR2b antibody each at a dose of 12-20 mg/kg, wherein the subsequent administrations are at a lower dose than the first administration; or
(b) an every two weeks (Q2W) regimen of the anti-FGFR2b antibody at a dose of greater than 10 mg/kg to no more than 20 mg/kg, and one week after the first administration of the anti-FGFR2b antibody, administering a single subsequent administration of the anti-FGFR2b antibody at a dose of 5-10 mg/kg.

2. The method of claim 1, wherein the solid tumor is selected from the group consisting of: squamous cancer, ER− PR− HER2/neu− (“triple-negative”) breast cancer, pancreatic ductal adenocarcinoma, intrahepatic cholangiocarcinoma, colorectal adenocarcinoma, gynecological malignancy, and lung adenocarcinoma.

3. (canceled)

4. The method of claim 1, wherein the anti-FGFR2b antibody monotherapy is administered as a second line or beyond therapy for the solid tumor.

5. The method of claim 2, wherein the squamous cancer is head and neck cancer or squamous esophageal cancer.

6. The method of claim 2, wherein the squamous cancer is head and neck squamous cell cancer.

7. The method of claim 2, wherein the gynecological malignancy is selected from the group consisting of ovarian epithelial cancer, endometrial cancer, and cervical cancer.

8. The method of claim 2, wherein the squamous cancer is post platinum-based chemotherapy and/or post-PD-1 inhibitor.

9. The method of claim 2, wherein the triple negative breast cancer is post chemotherapy, post-PARPi (if BRCA-mutated), post-PD-1 inhibitor therapy, and/or post-anti-trop-2 therapy.

10. The method of claim 2,
wherein the pancreatic ductal adenocarcinoma is post-platinum based chemotherapy,
wherein the intrahepatic cholangiocarcinoma is post-platinum based chemotherapy and post-targeted therapy, if eligible for targeted therapy, and/or
wherein the colorectal adenocarcinoma is post-bevacizumab therapy, post-oxaliplatin-based chemotherapy, post-irinotecan-based chemotherapy, and/or post-additional prior therapy based on RAS, BRAF, and dMMR/MSI-H status.

11. The method of claim 2, wherein the gynecological malignancy is post platinum-based chemotherapy, and/or is platinum chemotherapy resistant.

12. The method of claim 2, wherein the cells of the solid tumor overexpress FGFR2b mRNA or protein, or comprise an FGFR2 gene amplification.

13. The method of claim 1, wherein the solid tumor overexpresses FGFR2b as determined by immunohistochemistry (IHC).

14. The method of claim 13, wherein cells of the solid tumor are positive for FGFR2b as determined by IHC.

15. The method of claim 13, wherein cells of the solid tumor exhibit 2+ and/or 3+ FGFR2b staining as determined by IHC.

16. The method of claim 1, wherein (a) the first administration of the anti-FGFR2b antibody is at a dose of greater than 20 mg/kg to no more than 25 mg/kg, followed two weeks after the first administration and Q2W thereafter by subsequent administrations of the anti-FGFR2b antibody each at a dose of 12-17 mg/kg.

17. The method of claim 1, wherein (a) the first administration of the anti-FGFR2b antibody is at a dose of 22-25 mg/kg, followed two weeks after the first administration and Q2W thereafter by subsequent administrations of the anti-FGFR2b antibody each at a dose of 12-17 mg/kg.

18. The method of claim 17, wherein (a) the first administration of the anti-FGFR2b antibody is at a dose of about 22 mg/kg, followed two weeks after the first administration and Q2W thereafter by subsequent administrations of the anti-FGFR2b antibody each at a dose of about 15 mg/kg.

19. The method of claim 1, wherein (b) the Q2W regimen of the anti-FGFR2b antibody is at a dose of 12-17 mg/kg, and the subsequent single administration of the anti-FGFR2b antibody one week after the first administration of the anti-FGFR2b antibody is at a dose of 7-8 mg/kg.

20. The method of claim 19, wherein (b) the Q2W regimen of the anti-FGFR2b antibody is at a dose of about 15 mg/kg, and the subsequent single administration of the anti-FGFR2b antibody one week after the first administration of the anti-FGFR2b antibody is at a dose is about 7.5 mg/kg.

21. The method of claim 1, wherein the anti-FGFR2b antibody is administered intravenously.

22. The method of claim 1, wherein the anti-FGFR2b antibody comprises:
a heavy chain variable region comprising a heavy chain complementarity determining region (HCDR) 1 of SEQ ID NO: 6, a HCDR2 of SEQ ID NO: 7, and a HCDR3 of SEQ ID NO: 8; and
a light chain variable region comprising a light chain complementarity determining region (LCDR) 1 of SEQ ID NO: 9, a LCDR2 of SEQ ID NO: 10, and a LCDR3 of SEQ ID NO: 11.

23. The method of claim 1, wherein the anti-FGFR2b antibody is afucosylated.

24. The method of claim 20, wherein the heavy chain variable region comprises an amino acid sequence at least 95% identical to SEQ ID NO: 4, and wherein the light chain variable region comprises an amino acid sequence at least 95% identical to SEQ ID NO: 5.

25. The method of claim 24, wherein the heavy chain variable region comprises the amino acid sequence of SEQ ID NO: 4, and wherein the light chain variable region comprises the amino acid sequence of SEQ ID NO: 5.

26. The method of claim 25, wherein the anti-FGFR2b antibody comprises the heavy chain of SEQ ID NO: 1, and the light chain of SEQ ID NO: 2, and wherein the anti-FGFR2b antibody lacks fucose at Asn297 (EU numbering).

27. The method of claim 1, wherein the anti-FGFR2b antibody is bemarituzumab.

28. The method of claim 27, wherein the bemarituzumab is administered intravenously, wherein (a) the first administration is at a dose of greater than 20 mg/kg to no more than 25 mg/kg, followed two weeks after the first administration and Q2W thereafter by subsequent administrations of the bemarituzumab each at a dose of 12-17 mg/kg.

29. The method of claim 27, wherein the bemarituzumab is administered intravenously, wherein (a) the first administration is at a dose of 22-25 mg/kg, followed two weeks after the first administration and Q2W thereafter by subsequent administrations of the bemarituzumab each at a dose of 12-17 mg/kg.

30. The method of claim 27, wherein the bemarituzumab is administered intravenously, wherein (a) the first administration is at a dose of about 22 mg/kg, followed two weeks after the first administration and Q2W thereafter by subsequent administrations of the bemarituzumab each at a dose of about 15 mg/kg.

31. The method of claim 27, wherein the bemarituzumab is administered intravenously, wherein (b) the Q2W regimen of the bemarituzumab is at a dose of 12-17 mg/kg, and the subsequent single administration of the bemarituzumab one week after the first administration of the bemarituzumab is at a dose of 7-8 mg/kg.

32. The method of claim 27, wherein the bemarituzumab is administered intravenously, wherein (b) the Q2W regimen of the bemarituzumab is at a dose of about 15 mg/kg, and the subsequent single administration of the bemarituzumab one week after the first administration of the bemarituzumab is at a dose of about 7.5 mg/kg.

33. The method of claim 1, wherein the solid tumor is PD-L1 positive, as determined by IHC staining.
